(19) United States
(12) Patent Application Publication
(10) Pub. No.: US 2009/0028831 A1
(43) Pub. Date: Jan. 29, 2009

(54) STEM CELL REGULATOR, COMPOSITIONS AND METHODS OF USE

(75) Inventors: Gary Van Zant, Lexington, KY (US); Amanda Waterstrat, Georgetown, KY (US)

Correspondence Address: CROWELL & MORING LLP;INTELLECTUAL PROPERTY GROUP, P.O. BOX 14300, WASHINGTON, DC, 20044-4300, US

(73) Assignee: UNIVERSITY OF KENTUCKY RESEARCH FOUNDATION, Lexington, KY (US)

(21) Appl. No.: 11/781,533

(22) Filed: Jul. 23, 2007

(51) Int. Cl. A61K 35/00; A61P 43/00; C12N 5/06; C12Q 1/68; G01N 33/53

(52) U.S. Cl. 424/93.7; 435/383; 435/404; 435/6; 435/7.1

(57) ABSTRACT

The disclosure provides methods and compositions comprising Slit2 agonists and antagonists useful for modulating hematopoietic stem cell (HSC) proliferation and growth. In some aspects, the disclosure provides methods and compositions for stimulating HSC proliferation in vivo, ex vivo, or in vitro. In other aspects, the disclosure provides methods and compositions for inhibiting HSC proliferation.

### STATEMENTS REGARDING FEDERALLY SPONSORED RESEARCH

The invention was funded in part by Grant Nos. R01 AG024950, R01AG022859 and RO1 AG16653 awarded by the National Institutes of Health. The government has certain rights in the invention.

### TECHNICAL FIELD

The disclosure relates generally to compositions and methods useful for modulating stem cell growth in vitro and in vivo, as well as diagnostics useful for identifying cell proliferative disorders.

### BACKGROUND

The mammalian body is composed of several lineage-committed cells that give rise to the many tissues of a mammalian body. Despite the diversity of the nature, morphology, characteristics and function of such lineage committed cells, it is presently believed that most, if not all, the lineage committed cells are derived from various stem cells that give rise to one or more of the lineage committed cells of the mammalian body. Such stem cells constitute only a small percentage of the total number of cells present in the body and can vary depending up their relative commitment to a particular cell type.

### SUMMARY

The disclosure provides a composition comprising a basal medium and a Slit2 polypeptide or agonist thereof. In some aspect, the composition further comprises serum. In other aspect, the composition further comprises amino acids. In yet other aspect, the composition further comprises a reducing agent. The composition can further comprise antibiotics and/or fungicides. The composition can further comprise a pyruvate salt, L-gluatmine.

The disclosure also provides a composition comprising basal medium supplemented with serum, non-essential amino acids, an anti-oxidant, a reducing agent, growth factors, a pyruvate salt and a Slit2 polypeptide, homolog or variant thereof.

The disclosure provides a kit comprising a basal medium composition and a Slit2 polypeptide, homolog or variant thereof.

The disclosure also provides a method of culturing stem cells, comprising contacting the stem cells with a composition of described herein comprising a Slit2 polypeptide, homolog, or variant, wherein the stem cells grow and proliferate.

The disclosure also provides a method of treating a hematopoietic disease or disorder associated with reduced hematopoietic cells, comprising administering to a subject a Slit2 agonist, wherein the Slit2 agonist promotes hematopoietic stem cell proliferation and growth in the subject.

The disclosure also provides a method of treating a hematopoietic disease or disorder associated with reduced hematopoietic cells comprising isolating hematopoietic stem cells (HSCs); culturing the HSCs in the presence of a Slit2 agonist under conditions wherein the HSCs proliferate a grow to obtain an expanded HSC population; and administering the expanded HSC population to the subject.

The disclosure provides a method of treating a cell proliferative disorder or disease in a subject, wherein the cell proliferative disorder or disease comprise hematopoietic cells, the method comprising contact the subject with a Slit2 antagonist, wherein the Slit2 antagonist reduces the biological activity or expression of Slit2.

The disclosure also provides a method for diagnosing a cell proliferative disease or disorder comprising measuring an amount of a Slit2 polypeptide or polynucleotide in a sample; comparing the amount in the sample to a control amount, wherein an increase relative to the control is indicative of a cell proliferative disease or disorder.

The disclosure provides a pharmaceutical composition comprising a Slit2 agonist and a pharmaceutically acceptable carrier.

The disclosure also provides a pharmaceutical composition comprising a Slit2 antagonist and a pharmaceutically acceptable carrier.

### BRIEF DESCRIPTION OF DRAWINGS

FIG. 1 shows hematopoietic hierarchy and cell derivation.

FIG. 2A-B depict stem cell loss due, for example, to chemotherapeutics, age and disease.

FIG. 3 shows a linkage analysis and identification of proximal chromosome 5 as associated with stem cell number in mice.

FIG. 4 shows a schematic representation of backcrosses between B6 and D2 mice to generate two congenic mouse strains in which the chromosome 5 QTL region was exchanged between the two strains. The resulting congenic strains were used to verify the linkage and to harvest stem cells for gene expression profiling by microarray analysis.

FIG. 5 is a graph showing stem cell numbers B6, D2 and congenic mouse strains. The differences in stem cell number between congenic and parental strains verify that the region on proximal chromosome 5 contains a gene or genes that regulate hematopoietic stem cell number in mice.

FIG. 6 is a schematic depicting the results of gene expression profiling by microarray analysis and subsequent validation by quantitative RT-PCR.

FIG. 7 shows the level of expression of Slit2 mRNA in HSCs obtained from B6, D2, and congenic mice, measured by RT-PCR.

FIG. 8 is a table showing of the relationship between stem cell number and Slit2 expression in mice of the B6 or D2 genotype in the chromosome 5 QTL region. The B6 genotype is associated with low expression of Slit2 and low stem cell number while the D2 genotype is associated with high expression of Slit2 and high stem cell number.

FIG. 9 shows a graph of Slit2 mRNA, measured by RT-PCR, in bone marrow cell fractions of B6 and D2 mice. These results demonstrate that high expression of Slit2 is unique to the stem cell population of D2 mice.

FIG. 10 is a graph showing Slit2 expression measured by RT-PCR.

FIG. 11 shows Slit2 expression, measured by RT-PCR, and HSC cycling in bone marrow cells of B6 mice following 5-FU injection.

FIG. 12 shows a basic structure of Slit2.

### DETAILED DESCRIPTION

As used herein and in the appended claims, the singular forms “a,” “and,” and “the” include plural referents unless the context clearly dictates otherwise. Thus, for example, reference to “a cell” includes a plurality of such cells and reference to “the polypeptide” includes reference to one or more polypeptides known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Identification of a readily available source of stem cells and the modulation of stem cell proliferation both in vitro and in vivo is important based upon restrictions recently placed on the use of federal funding for stem cell research. The disclosure provides Slit2 biological factors (e.g., polypeptides, inhibitors, antagonists and agonists), methods, and compositions useful for propagating stem cells, including hematopoietic stem cells in culture, modulating (e.g., stimulating) stem cell growth and proliferation in vivo, and for expansion of stem cells ex vivo. Furthermore, the disclosure provides methods of promoting stem cell proliferation in the hematopoietic tissue of a subject being treated for a cell proliferative disorder, such as cancer. In such instances the administration of a Slit2 biological factor of the disclosure alone or in a pharmaceutical carrier can be used to treat a decrease in hematopoietic cells including stem cells in the subject. In addition, the disclosure provides methods of treating a cell proliferative disorder by modulating the production or activity of a Slit2 polypeptide.

Slit2 is a member of the Slit family of “neurological” migratory cues. Slit protein family members have been shown to be expressed by midline cells and endothelial cells and functions as a repellent in axon guidance (Kidd, T. et al. Cell 92:201-215 (1998); Brose, K. et al. Cell 96:795-806 (1999); Li, H. S. et al. Cell 96:807-818 (1999)) and branching (Wang, K.-H. et al. Cell 96:771-784 (1999); Whitford, K. L. et al. Neuron 33:47-61 (2002)), neuronal migration (Wu, W. et al. Nature 400:331-336 (1999)), and as an endogenous inhibitor for leukocyte chemotaxis (Wu, J. Y. et al. Nature 410:948-952 (2001)). Currently, there are three slit genes, slit1, 2 and 3, known in mammals. In addition, here is also a slit homologue called Slit-like 2. Their expression outside the nervous system has been found in rodents (Holmes, G. P. et al. Mech. Dev. 79:57-72 (1998); Piper, M. et al. Mech. Dev. 94: 213-217 (2000)). For example, mRNAs for Slit2 and Slit3 have been found in rat endothelial cells (Wu, J. Y. et al. Nature 410:948-952 (2001)). Slit homologs have been found in humans.

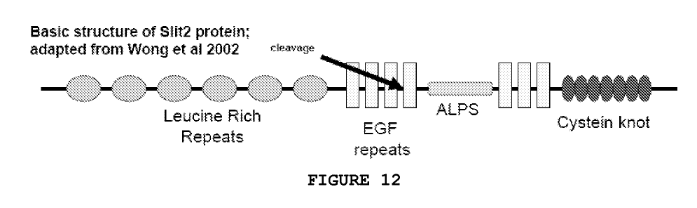

Slits are large ECM glycoproteins of ˜200 kDa, comprising, from their N terminus to their C terminus, a stretch of four leucine rich repeats, seven to nine EGF repeats, and a domain, named ALPS (for “agrin, laminin, perlecan, slit”), LNS (for “laminin, neurexin, slit”), or laminin G-like (LG) module (see, e.g., FIG. 12). Full-length hSlit2 is proteolytically processed into 140 kDa N-terminal and 55-60 kDa C-terminal fragments in cell culture and in vivo, typically between the 5 and 6 EGF repeat. The C-terminal domain of Slit is distantly related to cystine-knot domains of dimeric growth factors, such as TGF-β. Drosophila Slit appears to be similarly processed in vitro and in vivo. The heart of the Slit LRRs is characterized by the sequence LX₁X₂LX₃LX₄X₅N: the leucine side chains are buried in the hydrophobic core, while residues X₁-X₅ are exposed on the concave face of the domain and participate in ligand binding in other LRR proteins. There is evidence that the different Slit2 fragments have different functional activities in vivo. The purification of a DRG axon elongation- and branch-promoting activity suggested that the N-terminal fragment of Slit2, is capable of stimulating elongation and branching.

A number of receptors are known for Slit2 including members of the Robo family of protein receptors. Slit proteins are high-affinity ligands of the heparan sulfate proteoglycan glypican-1 (Liang et al., 1999; Ronca et al., 2001). Genetic and biochemical studies provide strong evidence that Slit proteins are ligands for the repulsive guidance transmembrane receptor Roundabout (Robo). Currently, there are four robe genes, robo1, robo2, rig-1 and robo4, known in mammals. Their expression outside the nervous system has been found in rodents (Holmes, G. P. et al. Mech. Dev. 79:57-72 (1998); Piper, M. et al. Mech. Dev. 94: 213-217 (2000)). For example, Robo1 RNA is found in mouse leukocytes (Wu, J. Y. et al. Nature 410:948-952 (2001)). Further, human endothelial cells express Robo4 (Huminiecki, L. et al. Genomics. 79:547-552 (2002)).

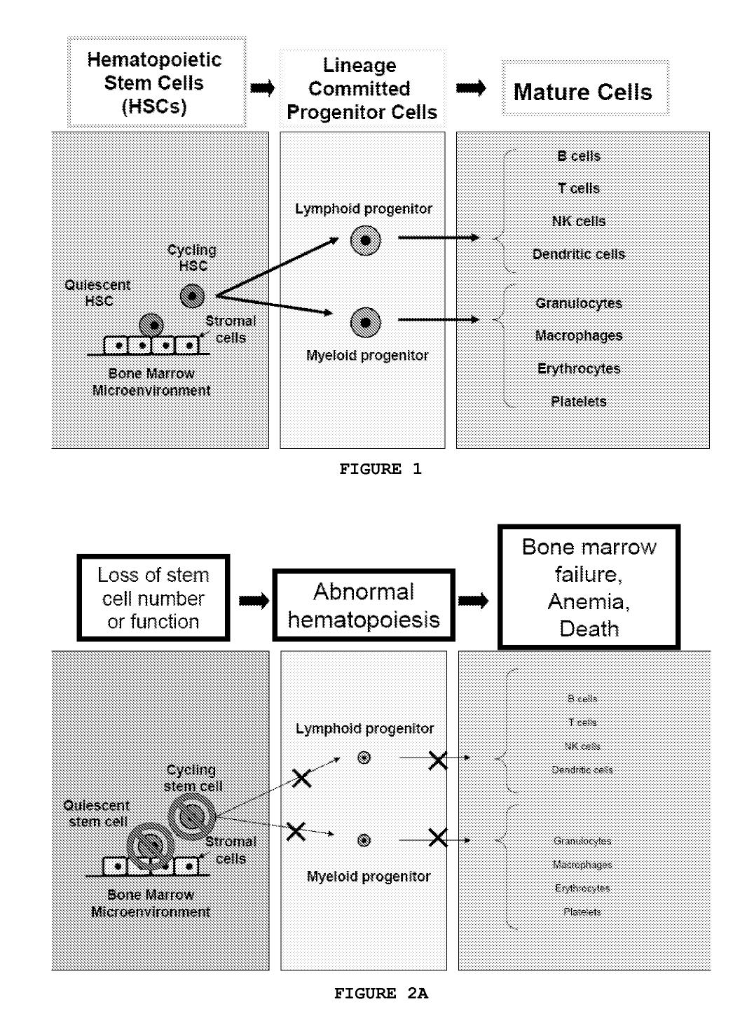

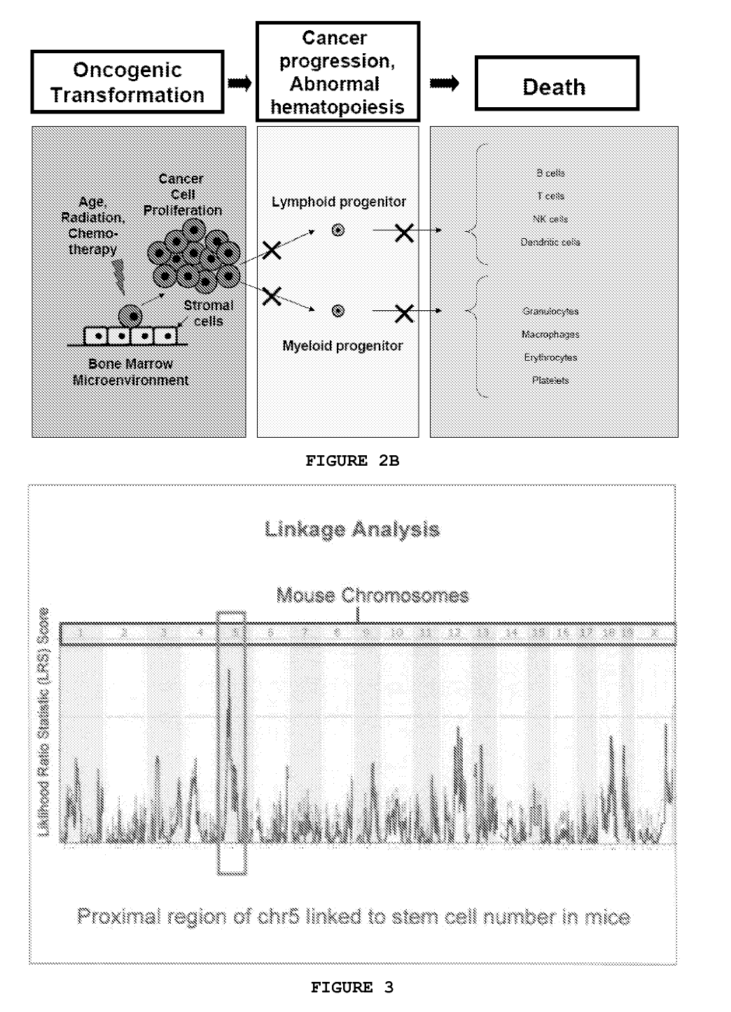

Slit2 polypeptides, as described further herein, have the ability to stimulate growth of hematopoietic stem cells (HSCs). HSCs are capable of maturing to erythroid, megakaryocyte, granulocyte, lymphocyte, and macrophage cells (FIG. 1). Many chemotherapeutic treatments (e.g., chemotherapeutic drugs and radiation therapies) deplete a subject of hematopoietic cells including hematopoietic stem cells (FIG. 2). The compositions and methods of the disclosure can be used in vivo to stimulate HSC proliferation and growth or be used in vitro or ex vivo to increase a population of HSCs in culture or prior to implantation. In one aspect, stromal cells are engineered to over express Slit2 in culture to facilitate HSC stimulation under co-culture conditions or to obtain conditioned media comprising a Slit2 from the stromal cells for use in culturing HSCs.

Hematopoietic stem cell transplantation (HSCT), of cells either derived from the bone marrow or peripheral blood, is used in the field of hematology and oncology. Such methods are used with diseases of the blood, bone marrow, or certain types of cancer.

Stem cell grafts/transplantation have included both allogeneic and autologous cells. In addition, the delivery of stem cells has included the stem cells in combination with various factors that assist in promoting stem cell proliferation and growth or committed cell growth and propagation. For example, stem cell growth factors GM-CSF and G-CSF are included with transplantation as well as ex vivo and in vitro stem cell cultures.

There is a group of stem cell disorders which are characterized by a reduction in functional marrow mass due to toxic, radiation-induced, or immunologic injury and which may be treatable with a Slit2 composition (e.g., a Slit2 polypeptide or polynucleotide). Aplastic anemia is a stem cell disorder in which there is a fatty replacement of hematopoietic tissue and pancytopenia. Slit2 can enhance hematopoietic proliferation and thus can be useful in treating aplastic anemia. Steel mice can be used as a model of human aplastic anemia (Jones, Exp. Hematol., 11, 571-580, 1983). Paroxysmal nocturnal hemoglobinuria (PNH) is a stem cell disorder characterized by formation of defective platelets and granulocytes as well as abnormal erythrocytes.

There are many diseases which are treatable with a Slit2 agent. These include the following: myelofibrosis, myelosclerosis, osteopetrosis, metastatic carcinoma, acute leukemia, multiple myeloma, Hodgkin's disease, lymphoma, Gaucher's disease, Niemann-Pick disease, Letterer-Siwe disease, refractory erythroblastic anemia, Di Guglielmo syndrome, congestive splenomegaly, Hodgkin's disease, Kala azar, sarcoidosis, primary splenic pancytopenia, miliary tuberculosis, disseminated fungus disease, Fulminating septicemia, malaria, vitamin B₁₂ and folic acid deficiency, pyridoxine deficiency, Diamond Blackfan anemia, hypopigmentation disorders such as piebaldism and vitiligo.

Enhancement of growth in non-hematopoietic stem cells such as primordial germ cells, neural crest derived melanocytes, commissural axons originating from the dorsal spinal cord, crypt cells of the gut, mesonephric and metanephric kidney tubules, and olfactory bulbs is of benefit where specific tissue damage has occurred to these sites. Slit2 can also be useful during in vitro fertilization procedures or in treatment of infertility states. Slit2 can be useful for treating intestinal damage resulting from irradiation or chemotherapy.

There are stem cell myeloproliferative disorders such as polycythemia vera, chronic myelogenous leukemia, myeloid mataplasia, primary thrombocythemia, and acute leukemias which can be treatable with Slit2 polypeptide or polynucleotides, anti-SLIT2 antibodies and the like.

Most recipients of HSCTs are leukemia subjects or others who would benefit from treatment with high doses of chemotherapy or total body irradiation. Other subjects who receive stem cell transplants include pediatric cases where the patient has a hereditary enzyme deficiency such as severe combined immunodeficiency or congenital neutropenia. Children or adults with aplastic anemia have lost their stem cells after birth and may not require such high doses of chemotherapy and irradiation prior to a transplant. Other diseases or disorders that can be treated with stem cell transplants include thalassemia major, sickle-cell disease, myelodysplastic syndrome, neuroblastoma, lymphoma, Hodgkin's disease, and multiple myeloma.

Autologous HSCs can be isolated from the subject to be treated prior to any treatment that may destroy existing stem cells. The stem cells are then propagated and/or stored for later use. The stem cells are then returned to the subject after treatment with, for example, a chemotherapeutic or irradiation. Autologous transplants have the advantage of a lower risk of graft rejection and infection, since the recovery of immune function is rapid. There is little chance for graft-versus-host disease, since the donor and recipient are the same individual.

Allogeneic HSC are obtained from a donor that is not the ultimate recipient. Allogeneic HSC donors may have a tissue (HLA) type that matches the recipient. Matching is performed on the basis of variability at three or more loci of the (HLA) gene, and an increased match at these loci provide improved graft retention. Immunosuppressive medications can be administered to the recipient to mitigate graft-versus-host disease/rejection. Allogeneic transplant donors may be related (usually a sibling) or unrelated volunteers. Allogeneic transplants are also performed using umbilical cord blood as the source of stem cells.

In the case of bone marrow, the HSC are removed from a large bone of the donor, typically the pelvis, through a large needle that reaches the center of the bone. The technique is referred to as a bone marrow harvest and is performed under general anesthesia because hundreds of insertions of the needle are required to obtain sufficient material.

Peripheral blood stem cells are now the most common source of stem cells for therapy. They are collected from the blood through a process known as apheresis. The donor's blood is withdrawn through a sterile needle in one arm and passed through a machine that removes white blood cells. The red blood cells are returned to the donor. Umbilical cord blood is also a source of HSCs. Cord blood has a higher concentration of HSC than is normally found in adult blood. In one aspect of the disclosure, the administration (e.g., hourly, daily, weekly, or monthly) of a Slit2 polypeptide, homolog or variant alone or in combination with Granulocyte-colony stimulating factor can boost stem cell counts in the donor subject prior to harvest from bone or blood.

HSCs can be frozen for prolonged time periods (cryopreserved). To cryopreserve HSC a preservative such as DMSO is added and the cells cooled very slowly to prevent osmotic cellular injury during ice crystal formation. HSC may be stored for years in a cryofreezer which typically utilizes liquid nitrogen.

As used herein a “culture” means a population of hematopoietic stem cells grown in a medium comprising Slit2 and optionally passaged accordingly. A stem cell culture may be a primary culture (e.g., a culture that has not been passaged) or may be a secondary or subsequent culture (e.g., a population of cells which have been subcultured or passaged one or more times in the presence of a Slit2).

Hematopoietic stem cells can be isolated from a sample obtained from a mammalian subject. The subject can be any mammal (e.g., bovine, ovine, porcine, canine, feline, equine, primate), but is preferably a human. The sample of cells may be obtained from any of a number of different sources including, for example, bone marrow, fetal tissue (e.g., fetal liver tissue), peripheral blood, umbilical cord blood, and the like.

In order to obtain stem cells of the disclosure, it may be useful to isolate, separate, or remove the stem cells of the disclosure from the other cells with which they are normally present. For example, where the source of cells is from peripheral blood the stem cells can be separated or enriched from other cells (e.g., erythrocytes, platelets, monocytes, neutrophils, macrophages, and the like).

Various techniques may be employed to separate the stem cells by initially removing the stem cells of the disclosure from other cell types via marker expression characteristics and/or by removing cells of dedicated lineage from the stem cells in a similar manner. Antibodies (e.g., monoclonal antibodies) are particularly useful for identifying cell surface protein markers associated with particular cell lineages and/or stages of differentiation. The antibodies may be attached to a solid support (e.g., antibody-coated magnetic beads). Examples of commercially available antibodies that recognize lineage dependent markers include anti-AC133 (Miltenyi Biotec, Auburn, Calif.); anti-CD34 (Becton Dickinson, San Jose, Calif.), anti-CD31, anti-CD62E, anti-CD104, anti-CD106, anti-CDla, anti-CD14 (all available from Pharmingen, Hamburg, Germany); anti-CD144 and anti-CD-13 (Immunotech, Marseille, France). The clone P1H12 (Chemicon, Temecula, Calif.; Catalog Number MAB16985), produces an antibody that specifically reacts with P1H12 antigen (also known as CD146, MCAM, and MUC18).

Procedures for separation may include magnetic separation, using antibody-coated magnetic beads, affinity chromatography, cytotoxic agents joined to a monoclonal antibody, or such agents used in conjunction with a monoclonal antibody, e.g., complement and cytotoxins, and “panning” with antibody attached to a solid matrix (e.g., plate), or other convenient technique. Techniques providing accurate separation include fluorescence activated cell sorters, which can have varying degrees of sophistication, e.g., a plurality of color channels, low angle and obtuse light scattering detecting channels, and impedance channels. Conveniently, antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the stem cells.

Once stem cells have been isolated, they can be propagated in medium comprising Slit2 alone or with other factors that promote stem cell growth and proliferation (e.g., EGM2 containing the commercially available supplements from Clonetics Corp., e.g., IGF, EGF, FGF, and VEGF +15% FCS, conditioned medium from other cell types, such as stromal cells (e.g., stromal cells obtained from bone marrow, fetal thymus or fetal liver), medium containing growth factors associated with stem cell maintenance, coculturing with stromal cells, or medium comprising maintenance factors supporting the proliferation of stem cells, where the stromal cells may be, for example, allogeneic or xenogeneic or genetically engineered cells that express Slit2.

In another embodiment, the disclosure provides methods of establishing and/or maintaining populations of stem cells, or the progeny thereof, as well as mixed populations comprising both stem cells and hematopoietic-like progeny cells, and the populations of cells so produced. As with the stem cells of the disclosure, once a culture of hematopoietic-like cells or a mixed culture of stem cells and hematopoietic-like cells is established, the population of cells is mitotically expanded in vitro by passage in a Slit2 containing medium under conditions that promote growth and proliferation.

Once the stem cells of the disclosure have been established in culture, as described above, they may be maintained or stored in cell “banks” comprising either continuous in vitro cultures of cells requiring regular transfer, or, the cells can be cryopreserved.

Cryopreserved cells constitute a bank of cells, portions of which can be withdrawn by thawing and then used to produce a stem cell culture comprising stem cells, hematopoietic and/or hematopoietic-like cells, or hematopoietic tissue as needed. Banked cells can be thawed and cultured in a medium comprising Slit2 under conditions to propagate the stem cells. As described herein, the stem cells may be used to produce new hematopoietic tissue for use in a subject where the cells were originally isolated from that subject's own blood or other tissue (i.e., autologous cells). Alternatively, the cells of the disclosure may be used as ubiquitous donor cells to produce new hematopoietic tissue for use in any subject (i.e., heterologous cells).

Once established, a culture of stem cells may be used to produce hematopoietic-like progeny cells and/or hematopoietic cells capable of producing new hematopoietic tissue. Differentiation of stem cells to hematopoietic cells or hematopoietic-like cells, followed by the production of hematopoietic tissue therefrom, can be triggered by specific exogenous growth factors or by changing the culture conditions (e.g., the density) of a stem cell culture. Since the cells are naive, they can be used to reconstitute an irradiated subject and/or a subject treated with chemotherapy; or as a source of cells for specific lineages, by providing for their maturation, proliferation and differentiation into one or more selected lineages. Examples of factors that can be used to induce differentiation include erythropoietin, colony stimulating factors, e.g., GM-CSF, G-CSF, or M-CSF, interleukins, e.g., IL-1, -2, -3, -4, -5, -6, -7, -8, and the like, Leukemia Inhibitory Factory (LIF), Steel Factor (Stl), or the like, coculture with cardiac myocytes, or other lineage committed cells types to induce the stem cells into becoming committed to a particular lineage.

In vitro or ex vivo Slit2 expanded HSCs can be infused into the blood stream of subject in need thereof through an intravenous (i.v.) catheter. The HSC briefly circulate in the blood stream and then populate the subject's bone marrow where they grow and start to produce blood cells. The growth and expansion of the stem cells can be further promoted by the administration of Slit2 alone or in combination with other factors such as stem cell factor, GCSF, GM-CSF and the like to the subject prior to, during, or after administration of HSCs. Hematopoeitic stem cells have been documented to populate many different organs of the recipient, including the heart, liver, and muscle, a phenomenon known as stem cell plasticity.

A Slit2 agonist comprises an agent that promotes or facilitates Slit2 biological activity. For example, a Slit2 agonist can comprise a Slit2 polypeptide, homolog or variant; a small molecule agent that promotes Slit2 activity by inducing second messenger signaling, binding and activation of a receptor; an antibody that binds to an activates a Slit2 receptor; an agent that activates transcription of Slit2 polynucleotides (e.g., a constitutive promoter operably linked to an endogenous Slit2 polynucleotide; and a heterogeneous Slit2 polynucleotide linked to a regulatory sequence that promotes transcription in the cell.

Treatment of mammals with a Slit2 agonist can result in an increases in hematopoietic cells of both myeloid and lymphoid lineages. One of the hallmark characteristics of stem cells is their ability to differentiate into both myeloid and lymphoid cells (Weissman, Science, 241:58-62, 1988).

As used herein a Slit2 polypeptide refers to a polypeptide that contains or comprises an amino acid sequence as set forth in Table 1 and associated with the GenBank accession numbers (which are incorporated herein by reference); polypeptides having substantial homology or substantial identity to the sequences as set forth in the accession number; a polypeptide comprising a fragment of any of the polypeptides identified herein and having HSC proliferative effects; fragments of the polypeptides; and conservative variants thereof.

TABLE 1

| Definition | Accession |
|---|---|
| Ovis aries Slit2 mRNA, partial cds | EF627036 |
| Mus musculus slit homolog 2 (Drosophila) (Slit2), mRNA | NM_178804 |
| Homo sapiens slit homolog 2 (Drosophila) (SLIT2), mRNA | NM_004787 |
| Danio rerio slit homolog 2 (slit2), mRNA | NM_131735 XM_696894 |
| Bos taurus similar to SLIT2, transcript variant 3, mRNA | XM_001250451 |
| Bos taurus similar to SLIT2, transcript variant 2 mRNA | XM_001250408 |
| Bos taurus similar to SLIT2, transcript variant 4, mRNA. | XM_613831 |
| Canis familiaris slit2 mRNA for Slit2, partial cds. | AB194048 |
| Gallus gallus slit homolog 2, variant 2 (SLIT2), mRNA. | XM_001232065 |
| Gallus gallus slit homolog 2, variant 1 (SLIT2), mRNA. | XM_001232040 |
| Pan troglodytes slit homolog 2, variant 1(SLIT2), mRNA. | XM_001163176 |
| Pan troglodytes slit homolog 2, variant 2 (SLIT2), mRNA. | XM_001163236 |
| Pan troglodytes slit homolog 2, variant 5 (SLIT2), mRNA. | XM_001163374 |
| Pan troglodytes slit homolog 2, variant 4 (SLIT2), mRNA. | XM_001163334 |
| Pan troglodytes slit homolog 2, variant 6 (SLIT2), mRNA. | XM_001163410 |
| Pan troglodytes slit homolog 2, variant 3 (SLIT2), mRNA. | XM_001163294 |
| Pan troglodytes slit homolog 2, variant 7 (SLIT2), mRNA. | XM_001163449 |
| Homo sapiens cDNA highly similar SLIT2 (SLIL2) mRNA. | AK027326 |
| Homo sapiens slit homolog 2 mRNA, complete cds. | BC117190 |
| Rattus norvegicus slit homolog 2 (Slit2), mRNA. | XM_001057837 |
| Rattus norvegicus slit homolog 2 (Slit2), mRNA. | XM_346464 |
| Macaca mulatta slit homolog 2 (SLIT2), mRNA. | XR_012895 |
| Canis familiaris slit homolog 2 (SLIT2), mRNA. | XM_849750 |
| Macaca mulatta slit-like protein 2 (SLIT2), partial cds. | AY083584 |
| Mus musculus SLIT2 (Slit2) mRNA, complete cds. | AF144628 |

A polypeptide of the disclosure also encompasses an amino acid sequence that has a sufficient or a substantial degree of identity or similarity to a sequence set forth in any of the sequences associated with the Accession Nos. in Table 1 (e.g., SEQ ID NO:2). Substantially identical sequences can be identified by those of skill in the art as having structural domains and/or having biological activity in common with a Slit2 polypeptide. Methods of determining similarity or identity may employ computer algorithms such as, e.g., BLAST, FASTA, and the like.

In one aspect, a Slit 2 polypeptide comprises (i) a sequence as set forth in SEQ ID NO:2; (ii) a sequence having 80%, 85%, 90%, 95%, 98% or 99% or more identity to SEQ ID NO:2 and having Slit2 biological activity; (iii) a polypeptide encoded by SEQ ID NO:1; (iv) a polypeptide encoded by a fragment of SEQ ID NO:1 and having Slit2 biological activity; (v) a polypeptide encoded by a nucleic acid that hybridizes to a nucleic acid consisting of SEQ ID NO:1 and encodes a polypeptide having Slit2 biological activity; and (vi) fragments of any of the foregoing polypeptides having Slit2 biological activity. Slit2 biological activity includes, but is not limited to, the ability to promote stem cell (e.g., hematopoietic stem cell) proliferation and growth.

In one aspect, a Slit2 polynucleotide comprises (i) a nucleic acid encoding a polypeptide of SEQ ID NO:2; (ii) a sequence as set forth in SEQ ID NO:1; (iii) a fragment of SEQ ID NO:1 encoding SEQ ID NO:2 or a fragment thereof having Slit2 biological activity; (iv) a nucleic acid that hybridizes to a polynucleotide consisting of SEQ ID NO:1 from nucleotide 205 to 4791 and encoding a polypeptide that has Slit2 biological activity; (v) a nucleic acid that hybridizes to a polynucleotide consisting of SEQ ID NO:1 and encoding a polypeptide that has Slit2 biological activity; and (vi) any of the foregoing wherein T can be U.

Polypeptides derived from a Slit2 polypeptide of the disclosure by any type of alteration (e.g., insertions, deletions, or substitutions of amino acids; changes in the state of glycosylation of the polypeptide; refolding or isomerization to change its three-dimensional structure or self-association state; and changes to its association with other polypeptides or molecules) are also encompassed by the disclosure. Therefore, the polypeptides provided by the disclosure include polypeptides characterized by amino acid sequences similar to that set forth, for example, in accession numbers associated with Table 1 and in SEQ ID NO:2, but into which modifications are naturally provided or deliberately engineered. A polypeptide that shares biological activities in common with a Slit2 polypeptide comprises stem cell proliferative activity.

The disclosure provides both full-length and mature forms of Slit2 polypeptides. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides may be encoded by a single genetic locus if multiple mRNA forms are produced from that locus by alternative splicing or by the use of multiple translation initiation sites. The “mature form” of a polypeptide refers to a polypeptide that has undergone post-translational processing steps, if any, such as, for example, cleavage of the signal sequence or proteolytic cleavage to remove a prodomain. As described above, Slit2 is proteolytically processed to provide an N-terminal and C-terminal domain via proteolytic cleavage within the EGF repeats. Multiple mature forms of a particular full-length polypeptide may be produced, for example, by imprecise cleavage of the signal sequence, or by differential regulation of proteases that cleave the polypeptide. The mature form(s) of such polypeptide may be obtained by expression, in a suitable insect or mammalian cell or other host cell, of a polynucleotide that encodes the full-length polypeptide. The sequence of the mature form of the polypeptide may also be determinable from the amino acid sequence of the full-length form, through identification of signal sequences or protease cleavage sites. Thus, in one aspect, a functional domain of Slit2 polypeptides comprise the N-terminal (˜140-170 kDa) domain. The N-terminal domain has binding affinity to, for example, members of the Robo receptor family. In addition, the disclosure includes the C-terminal (˜40-60 kDa) domain comprising structural similarities to TGF-β. The Slit2 polypeptides of the disclosure also include polypeptides that result from post-transcriptional or post-translational processing events such as alternate mRNA processing which can yield a truncated but biologically active polypeptide. Also encompassed within the disclosure are variations attributable to proteolysis such as differences in the N- or C-termini upon expression in different types of host cells, due to proteolytic removal of one or more terminal amino acids from the polypeptide (generally from 1-5 terminal amino acids).

A polypeptide of the disclosure may be prepared by culturing transformed or recombinant host cells under culture conditions suitable to express a polypeptide of the disclosure. The resulting expressed polypeptide may then be purified from such culture using known purification processes and used in vitro or in vivo. The purification of the polypeptide may also include an affinity column containing agents which will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography. Alternatively, the polypeptide of the disclosure may also be expressed in a form that will facilitate purification. For example, it may be expressed as a fusion polypeptide, such as those of maltose binding polypeptide (MBP), glutathione-S-transferase (GST) or thioredoxin (TRX). Kits for expression and purification of such fusion polypeptides are commercially available from New England BioLab (Beverly, Mass.), Pharmacia (Piscataway, N.J.), and InVitrogen, respectively. The polypeptide can also be tagged with an epitope and subsequently purified by using a specific antibody directed to such epitope. Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous recombinant polypeptide. The polypeptide thus purified is substantially free of other mammalian polypeptides and is defined in accordance with the disclosure as an “substantially purified polypeptide”; such purified polypeptides include Slit2 polypeptide, fragment, variant, and the like. A polypeptide of the disclosure may also be expressed as a product of transgenic animals or insects, which are characterized by somatic or germ cells containing a polynucleotide encoding a polypeptide of the disclosure.

It is also possible to utilize an affinity column such as a monoclonal antibody generated against polypeptides of the disclosure, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the disclosure. In this aspect of the disclosure, proteins that bind a polypeptide of the disclosure (e.g., an anti-Slit2 antibody) can be bound to a solid phase support or a similar substrate suitable for identifying, separating, or purifying cells that express polypeptides of the disclosure on their surface. Adherence of, for example, an anti-Slit2 antibody to a solid phase surface can be accomplished by any means, for example, magnetic microspheres can be coated with these polypeptide-binding proteins and held in the incubation vessel through a magnetic field.

A polypeptide of the disclosure may also be produced by known conventional chemical synthesis. Methods for constructing the polypeptides of the disclosure by synthetic means are known to those skilled in the art. The synthetically-constructed polypeptide sequences, by virtue of sharing primary, secondary or tertiary structural and/or conformational characteristics with a native polypeptides may possess biological properties in common therewith, including biological activity.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Typically, the polypeptide of the disclosure is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Species homologues of Slit2 polypeptides and polynucleotides encoding the polypeptides are also provided by the disclosure. As used herein, a “species homologue” is a polypeptide or polynucleotide with a different species of origin from that of a given polypeptide or polynucleotide, but with significant sequence similarity to the given polypeptide or polynucleotide. Species homologues may be isolated and identified by making suitable probes or primers from polynucleotides encoding the polypeptides provided herein and screening a suitable nucleic acid source from the desired species. Alternatively, homologues may be identified by screening a genome database containing sequences (e.g., nucleic acid or amino acid sequence) from one or more species comprising a Slit2 of the disclosure. A number of Slit2 homologs are identified in Table 1. Genome databases are readily available for a number of species (e.g., on the world wide web (www) at tigr.org/tdb; genetics.wisc.edu; stanford.edu/.about.ball; hiv-web.lan1.gov; ncbi.nlm.nig.gov; ebi.ac.uk; and pasteur.fr/other/biology). The disclosure also encompasses allelic variants of Slit2 that are naturally-occurring alternative forms of such polypeptides and polynucleotides in which differences in amino acid or nucleotide sequence are attributable to genetic polymorphism.

Intermediate Sequence Search (ISS) can be used to identify closely related as well as distant homologs by connecting two proteins through one or more intermediate sequences. ISS repetitively uses the results of the previous query as new search seeds. Saturated BLAST is a package that performs ISS. Starting with a protein sequence, Saturated BLAST runs a BLAST search and identifies representative sequences for the next generation of searches. The procedure is run until convergence or until some predefined criteria are met. Saturated BLAST is available on the world wide web (www) at: bioinformatics.burnham-inst.org/xblast (see also, Li et al. Bioinformatics 16(12): 1105, 2000).

Fragments of the Slit2 polypeptides of the disclosure are encompassed by the disclosure and may be in linear form or cyclized using known methods (see, e.g., H. U. Saragovi, et al., Bio/Technology 10, 773 (1992); and R. S. McDowell, et al., J. Amer. Chem. Soc. 114:9245 (1992), both of which are incorporated by reference herein). Peptide fragments of Slit2 polypeptides of the disclosure, and polynucleotides encoding such fragments include amino acid or nucleotide sequence lengths that are at least 25% (typically at least 50%, 60%, or 70%, and commonly at least 80%) of the length of a Slit2 polypeptide or polynucleotide. Typically such sequences will have at least 60% sequence identity (more typically at least 70%-75%, 80%-85%, 90%-95%, at least 97.5%, or at least 99%, and most commonly at least 99.5%) with a Slit2 polypeptide or polynucleotide when aligned so as to maximize overlap and identity while minimizing sequence gaps. Also included in the disclosure are polypeptides, peptide fragments, and polynucleotides encoding them, that contain or encode a segment comprising at least 8 to 10, typically at least 20, at least 30, or most commonly at least 40 contiguous amino acids. Such polypeptides and fragments may also contain a segment that shares at least 70% (at least 75%, 80%-85%, 90%-95%, at least 97.5%, or at least 99%, and commonly at least 99.5%) with any such segment of any of the Slit family polypeptides, when aligned so as to maximize overlap and identity while minimizing sequence gaps. Visual inspection, mathematical calculation, or computer algorithms can determine the percent identity.

The polypeptides of the disclosure can be made by direct synthesis or by expression from cloned polynucleotide of the disclosure. Means for expressing cloned polynucleotides are described herein and are generally known in the art. The following considerations are recommended for the design of expression vectors used to express polynucleotides encoding the Slit polypeptides of the disclosure.

In another aspect of the disclosure, a polypeptide may comprise oligomers and various combinations of Slit2 polypeptide domains (e.g., repeat domains of Slit2). Accordingly, polypeptides of the disclosure and polynucleotides include those comprising or encoding two or more copies of a domain. Also included are recombinant polypeptides and the polynucleotides encoding the polypeptides wherein the recombinant polypeptides are “chimeric polypeptides” or “fusion polypeptides” and comprise a Slit2 polypeptide as set forth in the accession numbers above including SEQ ID NO:2 operatively linked to a second polypeptide. The second polypeptide can be any polypeptide of interest having an activity or function independent of, or related to, the function of a Slit2 polypeptide. For example, the second polypeptide can be a domain of a related but distinct member of the Slit2 family of polypeptides or can be a binding cognate or stromal cells or extracellular matrix material.

The term “operatively linked” is intended to indicate that the Slit2 polypeptide and the second polypeptide are fused in-frame to each other. The second polypeptide can be fused to the N-terminus or C-terminus of a Slit2 polypeptide. For example, in one embodiment, the fusion polypeptide is a GST-Slit2 fusion polypeptide in which the Slit2 polypeptide is fused to the C-terminus of the GST sequences. Such fusion polypeptides can facilitate the purification of recombinant Slit2 polypeptides. In another embodiment, the fusion polypeptide comprises a Slit2 polypeptide comprising a heterologous signal sequence at its N-terminus. In certain host cells (e.g., mammalian host cells), expression and/or secretion of a Slit2 polypeptide can be increased through use of a heterologous signal sequence. Such methods can be useful for recombinant feeder layers that express Slit2 in culture with an HSC. As another example, a Slit2 polypeptide or fragment thereof may be fused to a hexa-histidine tag to facilitate purification of bacterially expressed protein, or to a hemagglutinin tag to facilitate purification of protein expressed in eukaryotic cells. Further, fusion polypeptides can comprise, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG peptide in the presence of certain divalent metal cations, as described in U.S. Pat. No. 5,011,912, hereby incorporated by reference. The 4E11 hybridoma cell line has been deposited with the ATCC under accession no. HB9259. Monoclonal antibodies that bind the FLAG peptide are available from Eastman Kodak Co., Scientific Imaging Systems Division, New Haven, Conn.

Encompassed by the disclosure are oligomers or fusion polypeptides that comprise a Slit2 polypeptide. Oligomers that can be used as fusion partners can be in the form of covalently linked or non-covalently-linked multimers, including dimers, trimers, or higher oligomers. In an alternative embodiment the disclosure is directed to oligomers comprising multiple polypeptides joined via covalent or non-covalent interactions between peptide moieties fused to the polypeptides. Such peptides can be peptide linkers (spacers), or peptides that have the property of promoting oligomerization. Leucine zippers and certain polypeptides derived from antibodies are among the peptides that can promote oligomerization of the polypeptides attached thereto, as described in more detail below.

Typically a linker will be a peptide linker moiety. The length of the linker moiety is chosen to optimize the biological activity of the polypeptide having a Slit2 sequence and can be determined empirically without undue experimentation. The linker moiety should be long enough and flexible enough to allow a Slit2 moiety to freely interact with a substrate or ligand. The linker moiety is typically a peptide between about one and 30 amino acid residues in length, preferably between about two and 15 amino acid residues. Preferred linker moieties are—-Gly-Gly-, GGGGS (SEQ ID NO:3), (GGGGS)ₙ (SEQ ID NO:4), GKSSGSGSESKS (SEQ ID NO:5), GSTSGSGKSSEGKG (SEQ ID NO:6), GSTSGSGKSSEGSGSTKG (SEQ ID NO:7), GSTSGSGKPGSGEGSTKG (SEQ ID NO:8), or EGKSSGSGSESKEF (SEQ ID NO:9). Linking moieties are described, for example, in Huston, J. S., et al., PNAS 85:5879 (1988), Whitlow, M., et al., Protein Engineering 6:989 (1993), and Newton, D. L., et al., Biochemistry 35:545 (1996). Other suitable peptide linkers are those described in U.S. Pat. Nos. 4,751,180 and 4,935,233, which are hereby incorporated by reference. A DNA sequence encoding a desired peptide linker can be inserted between, and in the same reading frame as, DNA sequences of the disclosure, using any suitable conventional technique. For example, a chemically synthesized oligonucleotide encoding the linker can be ligated between the sequences. In particular embodiments, a fusion polypeptide comprises from two to four or more Slit2 polypeptides, separated by peptide linkers.

The Slit2 polypeptides of the disclosure can also include a localization sequence to direct the polypeptide to particular cellular sites by fusion to appropriate organellar targeting signals or localized host proteins. A polynucleotide encoding a localization sequence, or signal sequence, can be ligated or fused at the 5′ terminus of a polynucleotide encoding a Slit2 polypeptide such that the signal peptide is located at the amino terminal end of the resulting fusion polynucleotide/polypeptide. In eukaryotes, the signal peptide functions to transport a polypeptide across the endoplasmic reticulum. The secretory protein is then transported through the Golgi apparatus, into secretory vesicles and into the extracellular space or the external environment. Signal peptides include pre-pro peptides that contain a proteolytic enzyme recognition site.

The localization sequence can be a nuclear-, an endoplasmic reticulum-, a peroxisome-, or a mitochondrial-localization sequence, or a localized protein. Localization sequences can be targeting sequences that are described, for example, in “Protein Targeting”, chapter 35 of Stryer, L., Biochemistry (4th ed.). W.H. Freeman, 1995. Some important localization sequences include those targeting the nucleus (e.g., KKKRK (SEQ ID NO:10)), mitochondria (MLRTSSLFTRRVQP SLFRNILRLQST (SEQ ID NO:11)), endoplasmic reticulum (KDEL; (SEQ ID NO:12)), peroxisome (SKF), plasma membrane (CAAX (SEQ ID NO:13), CC, CXC, or CCXX (SEQ ID NO:14)), cytoplasmic side of plasma membrane (fusion to SNAP-25), or the Golgi apparatus (fusion to furin).

A chimeric or fusion polypeptide of the disclosure can be produced by standard recombinant molecular biology techniques. In one embodiment, polynucleotide fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide).

The disclosure further includes polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the disclosure in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase.

Additional variants within the scope of the disclosure include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide.

The disclosure also provides polynucleotides encoding Slit2 polypeptides. The term “polynucleotide” refers to a polymeric form of nucleotides of at least 10 bases in length. The nucleotides can be ribonucleotides, deoxyribonucleotides, or modified forms of either type of nucleotide. The term includes single and double stranded forms of DNA or RNA. DNA includes, for example, cDNA, genomic DNA, chemically synthesized DNA, DNA amplified by PCR, and combinations thereof. The polynucleotides of the disclosure include full-length genes and cDNA molecules as well as a combination of fragments thereof. The polynucleotides of the disclosure are preferentially homogeneic to the stem cell population to be cultured or contacted (e.g., from a human source for human stem cell use).

A polynucleotide of the disclosure will generally contain phosphodiester bonds, although in some cases, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A variety of references disclose such nucleic acid analogs, including, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

Other analogs include peptide nucleic acids (PNA) which are peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

As described above, the nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant polynucleotide molecule, which is incorporated into a vector, e.g., an expression vector; into an autonomously replicating plasmid or virus; or into the genomic DNA of a prokaryote or eukaryote, or which exists as a separate molecule (e.g., a cDNA) independent of other sequences.

A Slit2 polynucleotide of the disclosure (1) encodes a Slit2 polypeptide as described herein and with reference to the accession numbers in Table 1; (2) comprises a sequence as set forth in the accession numbers in Table 1 (which are incorporated herein by reference) or the sequence as set forth in SEQ ID NO:1; (3) comprises a sequence complementary to any of the foregoing sequences; (4) comprises a fragment of any of the foregoing that specifically hybridize to the polynucleotide of (2) or (3) under moderate to highly stringent conditions and which has stem cell proliferative effects; and (5) polynucleotides of (1), (2), (3), or (4) wherein T can also be U (e.g., RNA sequences). Also encompassed by the disclosure are homologs of a Slit2 polynucleotide of the disclosure. Degenerate polynucleotides comprising sequences that encode a Slit2 polypeptide can be obtained by “back-translation” from the amino acid sequences of the disclosure. The polymerase chain reaction (PCR) procedure can be employed to isolate and amplify a DNA sequence encoding an fibroin polypeptide or a desired combination of fibroin polypeptide fragments. Oligonucleotides that define the desired termini of a target DNA molecule are employed as 5′ and 3′ primers. Accordingly, fragments of the polynucleotides of the disclosure are useful as probes and primers to identify or amplify related sequence or obtain full-length sequences of a Slit2 of the disclosure. The oligonucleotides can additionally contain recognition sites for restriction endonucleases, to facilitate insertion of the amplified combination of DNA fragments into an expression vector. PCR techniques are known in the art (see, e.g., PCR Protocols: A Guide to Methods and Applications, Innis et al., eds., Academic Press, Inc. (1990)).

The disclosure also includes polynucleotides and oligonucleotides that hybridize under reduced stringency conditions, typically moderately stringent conditions, and commonly highly stringent conditions, to Slit2 polynucleotides described herein. The basic parameters affecting the choice of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, F. M. Ausubel et al., eds., John Wiley & Sons, Inc., sections 2.10 and 6.3-6.4, incorporated herein by reference), and can be readily determined by those having ordinary skill in the art based on, for example, the length and/or base composition of the polynucleotide. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target polynucleotide of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tₘ) of the hybrid, where Tₘ is determined according to the following equations. For hybrids less than 18 base pairs in length, Tₘ (° C.)=2(# of A+T bases)+4(# of G+C bases). For hybrids above 18 base pairs in length, Tₘ (° C.)=81.5+16.6(log₁₀ [Na+])+0.41(% G+C)−(600/N), where N is the number of bases in the hybrid, and [Na+] is the concentration of sodium ions in the hybridization buffer ([Na+] for 1×SSC=0.165M). Each such hybridizing nucleic acid has a length that is at least 25% (at least 50%, 60%, or 70%, and most commonly at least 80%) of the length of a polynucleotide of the disclosure to which it hybridizes, and has at least 60% sequence identity (at least 70%, 75%, 80%, 85%, 90%, 95%, 97.5%, or at least 99%, and typically at least 99.5%) with a polynucleotide of the disclosure to which it hybridizes.

“Conservatively modified variants” applies to both polypeptide and polynucleotide. With respect to particular polynucleotide, conservatively modified variants refer to codons in the polynucleotide which encode identical or essentially identical amino acids. Because of the degeneracy of the genetic code, a large number of functionally identical polynucleotides encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such variations are “silent variations,” which are one species of conservatively modified variations. Every polynucleotide sequence herein that encodes a polypeptide also describes every possible silent variation of the nucleic acid. One of skill will recognize that each codon in a polynucleotide (except AUG, which is ordinarily the only codon for methionine) can be modified to yield a functionally identical molecule. Accordingly, each silent variation of a nucleic acid that encodes a polypeptide is implicit in each described sequence.

The isolated polynucleotides of the disclosure may be operably linked to an expression control sequence such as the pMT2 or pED expression vectors disclosed in Kaufman et al., Nucleic Acids Res. 19:4485 (1991); and Pouwels et al. Cloning Vectors: A Laboratory Manual, Elsevier, N.Y., (1985, and Supplements), in order to produce a polypeptide of the disclosure recombinantly. Many suitable expression control sequences are known in the art. General methods of expressing recombinant polypeptides are also known and are exemplified in R. Kaufman, Methods in Enzymology 185:537 (1990).

For example, expression of the Slit2 protein can be performed in E. coli by inserting the polyncleotide encoding Slit2 into plasmid vector. The Slit2 protein-coding gene is inserted in such a manner as to be operably linked to a promoter. This promoter is joined with sequences derived from the lac operator of E. coli, which confers regulation by lactose or analogs (IPTG). The E. coli host strain BL21(DE3) contains a lambda prophage which carries a gene encoding bacteriophage T7 RNA polymerase. This gene is controlled by a promoter which is also regulated by lactose or analogs. In addition to the phage T7 promoter, the vectors pFP202 and pFP204 provide sequences which encode a C-terminal tail containing six consecutive histidine resdues appended to the Slit2 protein-coding sequences. This tail provides a means of affinity purification of the protein under denaturing conditions through its adsorption to resins bearing immobilized Ni ions.

In addition, a sequence encoding an appropriate signal peptide (native or heterologous) can be incorporated into expression vectors. The choice of signal peptide or leader can depend on factors such as the type of host cells in which the recombinant polypeptide is to be produced. Examples of heterologous signal peptides that are functional in mammalian host cells include the signal sequence for interleukin (IL)-7 (see, U.S. Pat. No. 4,965,195); the signal sequence for IL-2 receptor (see, Cosman et al., Nature 312:768, 1984); the IL4 receptor signal peptide (see, EP 367,566); the type I IL-1 receptor signal peptide (see, U.S. Pat. No. 4,968,607); and the type II IL-1 receptor signal peptide (see, EP 460,846). A signal peptide that is functional in the intended host cells promotes extracellular secretion of the polypeptide. The signal peptide is cleaved from the polypeptide upon secretion of a polypeptide from the cell. A polypeptide preparation can include a mixture of polypeptide molecules having different N-terminal amino acids, resulting from cleavage of the signal peptide at more than one site.

Established methods for introducing DNA into mammalian cells (e.g., feeder cell layers such as fibroblasts) have been described (Kaufman, R. J., Large Scale Mammalian Cell Culture, 1990, pp. 15-69). Additional protocols using commercially available reagents, such as Lipofectamine or Lipofectamine-Plus lipid reagent (Gibco/BRL), can be used to transfect cells (Felgner et al., Proc. Natl. Acad. Sci. USA 84:7413, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487, 1990, describes several selection schemes, such as dihydrofolate reductase (DHFR) resistance. A suitable strain for DHFR selection can be CHO strain DX-B11, which is deficient in DHFR (Urlaub et al., Proc. Natl. Acad. Sci. USA 77:4216, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector are selected on the basis of resistance to these compounds.

Suitable host cells for expression of the polypeptide include eukaryotic, insect and prokaryotic cells. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) (see, McMahan et al. EMBO J. 10: 2821, 1991), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it may be possible to produce the polypeptide in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous polypeptides. Potentially suitable bacterial strains include, for example, Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain the functional polypeptide. Such covalent attachments may be accomplished using known chemical or enzymatic methods. The polypeptide may also be produced by operably linking a polynucleotide of the disclosure to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), as well as methods described in Summers and Smith, Texas Agricultural Experiment Station Bulletin No. 1555 (1987), and Luckow and Summers, Bio/Technology 6:47 (1988), incorporated herein by reference. Cell-free translation systems could also be employed to produce polypeptides using RNAs derived from nucleic acid constructs disclosed herein. A host cell that comprises an isolated polynucleotide of the disclosure, preferably operably linked to at least one expression control sequence, is a “recombinant host cell”.

Also comprehended by the disclosure are pharmaceutical compositions comprising therapeutically effective amounts of polypeptide products of the disclosure together with suitable diluents, preservatives, solubilizers, emulsifiers, adjuvants and/or carriers useful in stem cell therapy. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent adsorption to surfaces, detergents (e.g., Tween 20, Tween 80, Pluronic F68, bile acid salts), solubilizing agents (e.g., glycerol, polyethylene glycol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol, complexation with metal ions, or incorporation of the material into or onto particulate preparations of polymeric compounds such as polylactic acid, polglycolic acid, hydrogels, etc. or into liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance of a Slit2 polypeptide. The choice of composition will depend on the physical and chemical properties of the protein having Slit2 activity. Controlled or sustained release compositions include formulation in lipophilic carriers (e.g., fatty acids, waxes, oils). Also comprehended by the disclosure are particulate compositions coated with polymers (e.g., poloxamers or poloxamines) and Slit2 coupled to antibodies directed to tissue-specific receptors, ligands or antigens or coupled to ligands of tissue-specific receptors. Other embodiments of the compositions of the disclosure incorporate particulate forms, protective coatings, protease inhibitors or permeation enhancers for various routes of administration, including parenteral, pulmonary, nasal and oral.

The disclosure also comprises compositions including one or more additional hematopoietic factors such as EPO, G-CSF, GM-CSF, CSF-1, IL-1, IL-2, IL-3, IL-4, IL-5, IL-6, IL-7, IL-8, IL-9, IL-10, IL-11, IGF-I, or LIF (Leukemic Inhibitory Factor) administered in combination (either simultaneously or sequentially) with a Slit2 polypeptide.

A Slit2 polypeptide of the disclosure may be “labeled” by association with a detectable marker substance (e.g., radiolabeled with 125, or biotinylated) to provide reagents useful in detection and quantification of Slit2 or its receptor bearing cells in solid tissue and fluid samples such as blood or urine.

Slit2 is useful for expanding hematopoietic progenitors in syngeneic, allogeneic, or autologous bone marrow transplantation. The use of hematopoietic growth factors has been shown to decrease the time for neutrophil recovery after transplantation (Donahue, et al., Nature, 321, 872-875, 1986; and Welte et al., J. Exp. Med., 165, 941-948, 1987). For bone marrow transplantation, the following three scenarios are used alone or in combination: a donor is treated with Slit2 alone or in combination with other hematopoietic factors prior to bone marrow aspiration or peripheral blood leukapheresis to increase the number of cells available for transplantation; the bone marrow is treated in vitro to activate or expand the cell number prior to transplantation; finally, the recipient is treated to enhance engraftment of the donor marrow.

Slit2 is useful for enhancing the efficiency of gene therapy based on transfecting (or infecting with a retroviral vector) hematopoietic stem cells. Slit2 permits culturing and multiplication of the hematopoietic progenitor cells which are to be transfected. The culture can be done with Slit2 alone or in combination with IL-6, IL-3, or both. Once tranfected, these cells are then infused in a bone marrow transplant into patients suffering from genetic disorders (Lim, Proc. Natl. Acad. Sci, 86, 8892-8896, 1989). Examples of genes which are useful in treating genetic disorders include adenosine deaminase, glucocerebrosidase, hemoglobin, and cystic fibrosis.

Slit2 can also be used for treatment of acquired immune deficiency (AIDS) or severe combined immunodeficiency states (SCID) alone or in combination with other factors. Slit2 therapy, for example, could be used to increase the level of circulating T-helper lymphocytes. In addition, Slit2 can be useful for combatting the myelosuppressive effects of anti-HIV drugs such as AZT.

Slit2 can be used for enhancing hematopoietic recovery after acute blood loss.

The subject disclosure also relates to antibodies that can bind to Slit2 or a Slit2 receptor. Methods of generating monoclonal and polyclonal antibodies are known in the art. Monoclonal antibodies are useful to improve the selectivity and specificity of diagnostic and analytical assay methods using antigen-antibody binding. Also, they are useful to neutralize or remove Slit2 from serum. A second advantage of monoclonal antibodies is that they can be synthesized by hybridoma cells in culture, uncontaminated by other immunoglobulins.

Receptor-specific antibodies can either prevent ligand binding and receptor activation as well as antibodies that recognize the receptor-ligand complex and, in some aspects, do not specifically recognize the unbound receptor or the unbound ligand. Likewise, included in the disclosure are neutralizing antibodies which bind the ligand and prevent binding of the ligand to the receptor, as well as antibodies which bind the ligand, thereby preventing receptor activation, but do not prevent the ligand from binding the receptor. Further included in the disclosure are antibodies which activate the receptor. These antibodies may act as receptor agonists, i.e., potentiate or activate either all or a subset of the biological activities of the ligand-mediated receptor activation. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111 (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996) (which are all incorporated by reference herein in their entireties).

Antibodies of the disclosure may be used, for example, to purify, detect, and target the polypeptides of the disclosure, including both in vitro and in vivo diagnostic and therapeutic methods. For example, the antibodies have utility in immunoassays for qualitatively and quantitatively measuring levels of the polypeptides of the disclosure in biological samples. See, e.g., Harlow et al., Antibodies: A Laboratory Manual, (Cold Spring Harbor Laboratory Press, 2nd ed. 1988); incorporated by reference herein in its entirety.

Yet another embodiment of the disclosure is directed to a method for treating or preventing a cell proliferative disorder associated with altered, (e.g., increased or decreased), expression or activity of a Slit2 polypeptide, the method comprising administering to a subject in need of such treatment an effective amount of an antagonist of a Slit2 polypeptide. Typically, the cell proliferative disorder is cancer and the antagonist of the Slit2 polypeptide is an anti-Slit2 polypeptide antibody, Slit2 binding oligopeptide, Slit2 binding organic molecule or antisense oligonucleotide. Effective treatment or prevention of the cell proliferative disorder may be a result of direct killing or growth inhibition of cells that express a Slit2 polypeptide or by antagonizing the cell growth potentiating activity of a Slit2 polypeptide.

The terms “cell proliferative disorder” and “proliferative disorder” refer to disorders that are associated with some degree of abnormal cell proliferation. In one embodiment, the cell proliferative disorder is cancer. “Tumor”, as used herein, refers to all neoplastic cell growth and proliferation, whether malignant or benign, and all pre-cancerous and cancerous cells and tissues.

Antagonists of the disclosure include, for example, binding and/or inhibitory antibodies, antisense nucleic acids, ribozymes or soluble forms of Slit2 receptor polypeptides (e.g., Robo receptor family domains) of the disclosure and can include oligomers (e.g., Fc fusion protein) of such domains. Antagonists of the disclosure may be employed in a composition with a pharmaceutically acceptable carrier, e.g., as described herein.

Yet another embodiment of the disclosure is directed to a method of binding an antibody, oligopeptide or small organic molecule to a cell that expresses a Slit2 polypeptide, wherein the method comprises contacting a cell that expresses a Slit2 polypeptide with said antibody, oligopeptide or small organic molecule under conditions which are suitable for binding of the antibody, oligopeptide or small organic molecule to said Slit2 polypeptide and allowing binding therebetween.

Other embodiments of the disclosure are directed to the use of (a) a Slit2 polypeptide, (b) a nucleic acid encoding a Slit2 polypeptide or a vector or host cell comprising that nucleic acid, (c) an anti-Slit2 polypeptide antibody, (d) a Slit2-binding oligopeptide, or (e) a Slit2-binding small organic molecule in the preparation of a medicament useful for (i) the therapeutic treatment or diagnostic detection of a cancer or tumor, or (ii) the therapeutic treatment or prevention of a cell proliferative disorder.

Another embodiment of the disclosure is directed to a method for inhibiting the growth of a cancer cell, wherein the growth of said cancer cell is at least in part dependent upon the growth potentiating effect(s) of a Slit2 polypeptide (wherein the Slit2 polypeptide may be expressed either by the cancer cell itself or a cell that produces polypeptide(s) that have a growth potentiating effect on cancer cells), wherein the method comprises contacting the Slit2 polypeptide with an antibody, an oligopeptide or a small organic molecule that binds to the Slit2 polypeptide or to a receptor for Slit2 (e.g., Robo1, 2, or 3), thereby antagonizing the growth-potentiating activity of the Slit2 polypeptide and, in turn, inhibiting the growth of the cancer cell. Preferably the growth of the cancer cell is completely inhibited. Even more preferably, binding of the antibody, oligopeptide or small organic molecule to the Slit2 polypeptide induces the death of the cancer cell. Optionally, the antibody is a monoclonal antibody, antibody fragment, chimeric antibody, humanized antibody, or single-chain antibody. Antibodies, Slit2 binding oligopeptides and Slit2 binding organic molecules employed in the methods of the disclosure may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and Slit2 binding oligopeptides employed in the methods of the disclosure may optionally be produced in CHO cells or bacterial cells.

Yet another embodiment of the disclosure is directed to a method of therapeutically treating a tumor in a mammal, wherein the growth of said tumor is at least in part dependent upon the growth potentiating effect(s) of a Slit2 polypeptide, wherein the method comprises administering to the mammal a therapeutically effective amount of an antibody, an oligopeptide or a small organic molecule that binds to the Slit2 polypeptide or to a Slit2 receptor, thereby antagonizing the growth potentiating activity of said Slit2 polypeptide and resulting in the effective therapeutic treatment of the tumor. Optionally, the antibody is a monoclonal antibody, antibody fragment, chimeric antibody, humanized antibody, or single-chain antibody. Antibodies, Slit2 binding oligopeptides and Slit2 binding organic molecules employed in the methods of the disclosure may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and oligopeptides employed in the methods of the disclosure may optionally be produced in CHO cells or bacterial cells.

The methods and compositions can be used alone or in combination with other neoplastic/cancer/leukemia therapies. For example, the methods and compositions of the disclosure can be used in combination with chemotherapeutic drugs such as, but not limited to, 5-fluorouracil (5FU), cytosine arabinoside, cyclophosphamide, cisplatin, carboplatin, doxyrubicin, etoposide, taxol, and alkylating agents. Furthermore, combinations of nucleic acid inhibitors may be used.

Slit2 proteins, analogues, derivatives, and subsequences thereof, Slit2 nucleic acids (and sequences complementary thereto), anti-Slit2 antibodies, have uses in diagnostics. Such molecules can be used in assays, such as immunoassays, to detect, prognoses, diagnose, or monitor various conditions, diseases, and disorders affecting Slit2 expression, or monitor the treatment thereof. In particular, such an immunoassay is carried out by a method comprising contacting a sample derived from a patient with an anti-Slit2 antibody under conditions such that immunospecific binding can occur, and detecting or measuring the amount of any immunospecific binding by the antibody. In a specific aspect, such binding of antibody, in tissue sections, can be used to detect aberrant Slit2 localization or aberrant (e.g., low or absent) levels of Slit2. In a specific embodiment, antibody to Slit2 can be used to assay in a patient tissue or serum sample for the presence of Slit2 where an aberrant level of Slit2 is an indication of a diseased condition. By “aberrant levels,” is meant increased or decreased levels relative to that present, or a standard level representing that present, in an analogous sample from a portion of the body or from a subject not having the disorder.

Thus, the disclosure provides a method of detecting a cell proliferative disorder in a sample from a subject by contacting a sample having, or suspected of having, a cell proliferative disorder with a reagent that binds to a Slit2-specific cell component and detecting binding of the reagent to the component; comparing the level of binding in the sample with the level of binding in control, wherein an increased level of binding of the reagent in the sample is indicative of a cell proliferative disorder.

As used herein, a “Slit2-specific cell component” includes, but is not limited to, RNA and DNA encoding an Slit2 protein, the Slit2 protein and fragments thereof, and Slit2 variants including translocations in Slit2 nucleic acids, truncations in the Slit2 gene or protein, changes in nucleotide or amino acid sequence relative to wild-type Slit2.

Thus, the Slit2 molecules can act as novel diagnostic targets and therapeutic agents for controlling one or more of cellular proliferative and/or differentiative disorders.

The immunoassays which can be used include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

Slit2 genes and related nucleic acid sequences and subsequences, including complementary sequences, can also be used in hybridization assays. Slit2 nucleic acid sequences, or subsequences thereof comprising about at least 8 nucleotides, can be used as hybridization probes.

Hybridization assays can be used to detect, prognose, diagnose, or monitor conditions, disorders, or disease states associated with aberrant changes in Slit2 expression and/or activity as described. In particular, such a hybridization assay is carried out by a method comprising contacting a sample containing nucleic acid with a nucleic acid probe capable of hybridizing to Slit2 DNA or RNA, under conditions such that hybridization can occur, and detecting or measuring any resulting hybridization.

In specific embodiments, diseases and disorders involving over-proliferation of cells can be diagnosed, or their suspected presence can be screened for, or a predisposition to develop such disorders can be detected, by detecting altered levels of Slit2 protein, Slit2 RNA, or Slit2 functional activity or by detecting mutations in Slit2 RNA, DNA or protein (e.g., translocations in Slit2 nucleic acids, truncations in the Slit2 gene or protein, changes in nucleotide or amino acid sequence relative to wild-type Slit2) that cause altered expression or activity of Slit2. By way of example, levels of Slit2 protein can be detected by immunoassay, levels of Slit2 RNA can be detected by hybridization assays (e.g., Northern blots, dot blots), translocations and point mutations in Slit2 nucleic acids can be detected by Southern blotting, RFLP analysis, PCR using primers that preferably generate a fragment spanning at least most of the Slit2 gene, sequencing of the Slit2 genomic DNA or cDNA obtained from the patient.

In another embodiment, diseases and disorders involving a deficiency in cell proliferation or in which cell proliferation is desirable for treatment, are diagnosed, or their suspected presence can be screened for, or a predisposition to develop such disorders can be detected, by detecting decreased levels of Slit2 protein, Slit2 RNA, or Slit2 functional activity, or by detecting mutations in Slit2 RNA, DNA or protein (e.g., translocations in Slit2 nucleic acids, truncations in the gene or protein, changes in nucleotide or amino acid sequence relative to wild-type Slit2) that cause decreased expression or activity of Slit2. By way of example, levels of Slit2 protein, levels of Slit2 RNA, Slit2 binding activity, and the presence of translocations or point mutations can be determined as described.

In using a monoclonal antibody for the in vivo detection of antigen, the detectably labeled monoclonal antibody is given in a dose that is diagnostically effective. The term “diagnostically effective” means that the amount of detectably labeled monoclonal antibody is administered in sufficient quantity to enable detection of the site having the Slit2 antigen for which the monoclonal antibodies are specific. The concentration of detectably labeled monoclonal antibody which is administered should be sufficient such that the binding to those cells having Slit2 is detectable compared to the background. Further, it is desirable that the detectably labeled monoclonal antibody be rapidly cleared from the circulatory system in order to give the best target-to-background signal ratio.

For in vivo diagnostic imaging, the type of detection instrument available is a major factor in selecting a given radioisotope. The radioisotope chosen must have a type of decay that is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 keV range, which may be readily detected by conventional gamma cameras.

For in vivo diagnosis, radioisotopes may be bound to immunoglobulin, either directly or indirectly, by using an intermediate functional group. Intermediate functional groups which often are used to bind radioisotopes that exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions that can be bound to the monoclonal antibodies of the disclosure are ¹¹¹In, ⁹⁷Ru, ⁶⁷ Ga, ⁶⁸Ga, ⁷²As, ⁸⁹Zr, and ²⁰¹Tl.

A monoclonal antibody useful in the method of the disclosure can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements that are particularly useful in such techniques include ¹⁵⁷Gd, ⁵⁵Mn, ¹⁶²Dy, ⁵²Cr, and ⁵⁶Fe.

The disclosure provides methods and compositions useful for propagating stem cells and hematopoietic and/or hematopoietic-like cells that result from such methods and compositions, methods of isolating and using such stem cells compositions comprising them, and cells derived from them. The stem cells of the disclosure find utility in gene therapy, tissue engineering, tissue generation, wound repair, diagnostics, as angiogenic agents, as vasculogenic agents, as agents for gene and protein delivery, and as therapeutics.

In one aspect, culture medium is provided that comprises a Slit2 polypeptide. The culture medium can be a basal medium. A basal medium refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves can not synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of basal media are known in the art of mammalian cell culture, such as Dulbecco's Modified Eagle Media (DMEM), Knockout-DMEM (KO-DMEM), and DMEM/F12, although any base medium that can be supplemented with bFGF, insulin, and ascorbic acid and which supports the growth of stem cells in a substantially undifferentiated state can be employed.

“Conditioned medium” refers to a growth medium comprising a Slit2 polypeptide that is further supplemented with soluble factors derived from cells cultured in the medium. Techniques for isolating conditioned medium from a cell culture are well known in the art. As will be appreciated, conditioned medium is typically cell-free. In this context, “cell-free” refers to a conditioned medium that contains fewer than about 10%, but typically fewer than about 5%, 1%, 0.1%, 0.01%, 0.001%, or 0.0001% than the number of cells per unit volume, as compared to the culture from which it was separated.

A “defined” medium refers to a biochemically defined formulation comprised solely of the biochemically-defined constituents including a Slit2 polypeptide. A defined medium may include solely constituents having known chemical compositions. A defined medium may also include constituents that are derived from known sources. For example, a defined medium may also include factors and other compositions secreted from known tissues or cells; however, the defined medium will not include the conditioned medium from a culture of such cells. Thus, a “defined medium” may, if indicated, include a particular compounds added to form the culture medium, up to and including a portion of a conditioned medium that has been fractionated to remove at least one component detectable in a sample of the conditioned medium that has not been fractionated. Here, to “substantially remove” one or more detectable components of a conditioned medium refers to the removal of at least an amount of the detectable, known component(s) from the conditioned medium so as to result in a fractionated conditioned medium that differs from an unfractionated conditioned medium in its ability to support the long-term substantially undifferentiated culture of stem cells. Fractionation of a conditioned medium can be performed by any method (or combination of methods) suitable to remove the detectable component(s), for example, gel filtration chromatography, affinity chromatography, immune precipitation, and the like.

A growth factor refers to a substance that is effective to promote the growth of stem cells and which, unless added to the culture medium as a supplement, is not otherwise a component of the basal medium. Growth factors include, but are not limited to, basic fibroblast growth factor (bFGF), acidic fibroblast growth factor (aFGF), epidermal growth factor (EGF), insulin-like growth factor-I (IGF-I), insulin-like growth factor-II (IGF-II), platelet-derived growth factor-AB (PDGF), and vascular endothelial cell growth factor (VEGF), activin-A, and bone morphogenic proteins (BMPs), insulin, cytokines, chemokines, morphogents, neutralizing antibodies, other proteins, and small molecules.

A “non-essential amino acid” refers to an amino acid species that need not be added to a culture medium for a given cell type, typically because the cell synthesizes, or is capable of synthesizing, the particular amino acid species. While differing from species to species, non-essential amino acids are known to include L-alanine, L-asparagine, L-aspartic acid, L-glutamic acid, glycine, L-proline, and L-serine.

A cell culture is “essentially serum-free” when it does not contain exogenously added serum. If the cells being cultured produce some or all of the components of serum, of if the cells to be cultured are derived from a seed culture grown in a medium that contained serum, the incidental co-isolation and subsequent introduction into another culture of some small amount of serum (e.g., less than about 1%) should not be deemed as an intentional introduction of serum.

A medium according to the disclosure comprises a Slit2 polypeptide or homolog or variant thereof. The medium may also include, without limitation, non-essential amino acids, an anti-oxidant, a reducing agent, growth factors, and a pyruvate salt. The base media may, for example, be Dulbecco's Modified Eagle Medium (DMEM), DMEM/F-12, or KO-DMEM, each supplemented with L-glutamine (e.g., including the dipeptide L-alanyl-L-glutamine (Invitrogen)), non-essential amino acids, and β-mercaptoethanol. A medium is typically sterilized (e.g., by filtration) prior to addition to a cell culture. The medium may also be supplemented with antibiotics and fungicides.

Exogenous growth factors may also be added to a medium according to the disclosure to assist in the maintenance of cultures of stem cells in a substantially undifferentiated state. Such factors and their effective concentrations can be identified as described elsewhere herein or using techniques known to those of skill in the art of culturing cells. Representative examples of growth factors useful in the compostitions and methods of the disclosure include bFGF, insulin, acidic FGF (aFGF), epidermal growth factor (EGF), insulin-like growth factor I (IGF-I), IGF-II, platelet-derived growth factor (PDGF), and vascular endothelial growth factor (VEGF), activin-A, bone morphogenic proteins (BMPs), forskolin, glucocorticords (e.g., dexamethasone), transferrins, and albumins.

Useful reducing agents include beta-mercaptoethanol. Other reducing agents such as monothioglycerol or dithiothreitol (DTT), alone or in combination, can be used to similar effect. Still other equivalent substances will be familiar to those of skill in the cell culturing arts.

Pyruvate salts may also be included in a medium according to the disclosure. Pyruvate salts include sodium pyruvate or another pyruvate salt effective maintaining and/or enhancing stem cell growth in a substantially undifferentiated state such as, for example, potassium pyruvate.

Other compounds suitable for supplementing a culture medium of the disclosure include nucleosides (e.g., adenosine, cytidine, guanosine, uridine, and thymidine) and nucleotides. Nucleosides and/or nucleotides can be included in a variety of concentrations.

Once isolated, the stem cells, can be cultured in a culture medium according to the disclosure that supports the substantially undifferentiated growth of stem cells using any suitable cell culturing technique.

The cell culture media of the disclosure and methods for growing stem cells in accordance with the disclosure will be seen to be applicable to all technologies for which stem cell lines are useful. For example, cells cultured based upon the media and methods of the disclosure can be used for screening to identify growth factors useful in culturing stem cells in an undifferentiated state, as well as compounds that induce such cells to differentiate toward a particular cell or tissue lineage. The disclosure also allows genetically modified stem cells to be developed, as well as the creation of new stem cell lines.

The disclosure also provides kits comprising a Slit2 polypeptide, homolog or variant thereof and various basal media compositions. The kits can be compartmentalized to maintain separation of the Slit2 polypeptide, homolog or variant until use at which point it can be added to the basal media.

Stem cells that can be propagated by methods and compositions of the disclosure express one or more markers associated with an hematopoietic stem cell phenotype and/or lack one or more markers associated with a differentiated cell (e.g., a cell having a reduced capacity for self-renewal, regeneration, or differentiation) and/or a cell of hematopoietic origin. A molecule is a “marker” of a desired cell type if it is found on a sufficiently high percentage of cells of the desired cell type, and found on a sufficiently low percentage of cells of an undesired cell type, that one can achieve a desired level of purification of the desired cell type from a population of cells comprising both desired and undesired cell types by selecting for cells in the population of cells that have the marker. A marker can be displayed on, for example, 30%, 35%, 40%, 45%, 50%, 55%, 60%, 65%, 70%, 75%, 80%, 85%, 90%, 95%, 99% or more of the desired cell type, and can be displayed on fewer than 50%, 45%, 40%, 35%, 30%, 25%, 20%, 15%, 10%, 5%, 1% or fewer of an undesired cell type.

The term “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew its line or to produce progeny cells which will differentiate into hematopoietic cells or hematopoietic-like cells; or a lineage-committed progenitor cell and its progeny, which is capable of self-renewal and is capable of differentiating into an hematopoietic cell. Unlike pluripotent stem cells, lineage-committed progenitor cells are generally considered to be incapable of giving rise to numerous cell types that phenotypically differ from each other. Instead, they give rise to one or possibly two lineage-committed cell types.

In one aspect, the disclosure provides isolated stem cells, individually or in populations. The term “isolated” or “purified” when referring to stem cells of the disclosure means cells that are substantially free of cells carrying markers associated with lineage dedication. In particular embodiments, the stem cells are at least 30%, 35%, 40%, 45%, 50%, 55%, 60%, 65%, 70%, 75%, 80%, 85%, 90%, 95% or 99% free of such contaminating cell types. In another embodiment, the isolated stem cells also are substantially free of soluble, naturally occurring molecules. As discussed more fully below, a substantially purified stem cell of the disclosure can be obtained, for example, by extraction (e.g., via density gradient centrifugation and/or flow cytometry) from a natural source such as a tissue or blood sample. Purity can be measured by any appropriate method. A stem cell of the disclosure can be 99%-100% purified by, for example, flow cytometry (e.g., FACS analysis), as discussed below.

In one embodiment, the disclosure provides methods and compositions that are useful for enriching stem cells and enriched stem cell compositions obtained therefrom. An “enriched population of stem cells” is one wherein stem cells of the disclosure have been partially separated from other cell types, such that the resulting population of stem cells has a greater concentration of stem cells than the original population of cells had. The enriched population of stem cells can have greater than about a 10-fold, 100-fold, 500-fold, 1,000-fold, 2,000-fold, 3,000-fold, 4,000-fold, 5,000-fold, 6,000-fold, 7,000-fold, 8,000-fold, 9,000-fold, 10,000-fold or greater concentration of stem cells than the original population had prior to separation. Stem cells of the disclosure can, for example, make up at least 5%, 10%, 15%, 20%, 35%, 30%, 35%, 40%, 45%, 50%, 55%, 60%, 65%, 70%, 75%, 80%, 85%, 90%, 95%, 99% or more of the enriched population of stem cells. The enriched population of stem cells may be obtained by, for example, selecting against cells displaying markers associated with differentiated cells, or other undesired cell types, and/or selecting for cells displaying markers associated with the stem cells of the disclosure, and/or by regenerating isolated stem cells in defined culture systems comprising Slit2 or other hematopoietic growth factors, as discussed herein.

In another embodiment, the disclosure provides cell lines of stem cells obtained from the methods and compositions described herein (e.g., culturing stem cells with Slit2 polypeptide or polynucleotide; or cells genetically modified to produced Slit2). As used herein a “cell line” means a culture of stem cells of the disclosure, or progeny cells thereof (e.g., hematopoietic and/or hematopoietic-like cells), that can be reproduced for an extended period of time, preferably indefinitely, and which term includes, for example, cells that are cultured, cryopreserved and re-cultured following cryopreservation.

The cells of the disclosure can be used to produce new hematopoietic tissue in vitro, which can then be implanted, transplanted or otherwise inserted into a site requiring hematopoietic tissue repair, replacement or augmentation in a subject. In a non-limiting embodiment, the stem cells of the disclosure are used to produce a three-dimensional tissue construct in vitro, which is then implanted in vivo. As an example of the production of three-dimensional tissue constructs, see U.S. Pat. No. 4,963,489, issued Oct. 16, 1990, to Naughton et al., which is incorporated herein by reference. For example, the hematopoietic stem cells or hematopoietic stem cells and hematopoietic and/or hematopoietic-like cells of the disclosure may be inoculated or “seeded” onto a three-dimensional framework or scaffold, and proliferated or grown in vitro to form a living hematopoietic tissue which can be implanted in vivo.

Therapeutic uses of the stem cells of the disclosure include transplanting the stem cells, stem cell populations, or progeny thereof into individuals to treat a variety of pathological states including diseases and disorders resulting from myocardial damage, circulatory or vascular disorders or diseases, as well as tissue regeneration and repair. Stem cells or stem cell populations (including genetically altered stem cells) are introduced into a subject in need of such stem cells or in need of the protein or molecule encoded or produced by the genetically altered cell. For example, in one embodiment, the stem cells can be administered to cancer patients who have undergone chemotherapy that have killed, reduced, or damaged hematopoietic stem cells, hematopoietic cells, or endothelium of a subject.

If the stem cells are derived from heterologous source compared to the recipient subject, concomitant immunosuppression therapy is typically administered, e.g., administration of the immunosuppressive agent cyclosporine or FK506. However, due to the immature state of the stem cells of the disclosure such immunosuppressive therapy may not be required. Accordingly, in one embodiment, the stem cells of the disclosure can be administered to a recipient in the absence of immunomodulatory (e.g., immunsuppressive) therapy. Alternatively, the cells can be encapsulated in a membrane, which permits exchange of fluids but prevents cell/cell contact. Transplantation of microencapsulated cells is known in the art, e.g., Balladur et al., 1995, Surgery 117:189-94, 1995; and Dixit et al., 1992, Cell Transplantation 1:275-79.

The cells may be introduced directly into the peripheral blood or deposited within other locations throughout the body, e.g., the spleen, pancreas, or on microcarrier beads in the peritoneum. For example, 10² to 10⁹ cells can be transplanted in a single procedure, and additional transplants can be performed as required.

Differentiation of the stem cells can be induced ex vivo, or alterantively may be induced by contact with tissue in vivo, (e.g., by contact with hematopoietic cells or cell matrix components). Optionally, a differentiating agent may be co-administered or subsequently administered to the subject to promote stem cell differentiation.

The following examples are offered to more fully illustrate the disclosure, but are not to be construed as limiting the scope thereof.

### EXAMPLES

Quantification of Hematopoietic Stem Cell Number (CAFC assay). Test populations of MBMCs were pooled from the femora of 3-6 mice/strain and plated onto confluent monolayers of FBMD-1 stromal support cells in 96-well tissue culture-treated plates established 10-14 days prior. Cells were seeded in threefold dilutions from 81,000 to 333 cells per well. Twenty replicate wells per dilution were evaluated and experiments were repeated a minimum of 3 times. Screening was performed at 7, 14, 21, 28, and 35 days for the presence of cobblestone areas. Later time points represent the appearance of increasingly primitive colony forming cells. The frequency of HSCs is determined by cobblestone areas counted on days 28 and 35. Cell frequency is calculated by maximum likelihood analysis based on Poisson distribution using the L-Calc software package from Stem Cell Technologies (Vancouver, B.C; Canada).

Linkage analysis. Discovery of the chromosome 5 QTL by linkage analysis was published by Geiger et. al. in 2001, and performed as described by de Haan and Van Zant in 1999. Briefly, hematopoietic stem cell (HSC) number was quantified by CAFC analsyis in 26 BXD recombinant inbred mouse strains. The set of trait values (HSC number/femur) for all 26 strains was correlated to genetic variation in these strains by a genome-wide scan for linked loci. At the time of discovery, the MapManagerQTb28 program, containing a BXD marker database of 319 marker loci, was employed for linkage analysis.

FIG. 3 displays the results of a comprehensive linkage analysis. In this linkage analysis, HSC number was collected for 36 BXD strains and both parental strains. This set of trait data was entered into a mapping program called WebQTL (http˜˜www.genenetwork.org/). This program correlates phenotype data to genotype data for a set of 3,795 markers typed across the genome of 88 BXD strains. This program provided mapping and also identified the proximal region of chromosome 5 as being linked to HSC number in BXD mice with a peak LRS score of 14.074 (9.96=suggestive, 16.20=significant) located at the 40 mBP position, and a 95% confidence interval ranging from 29-55 megabases.

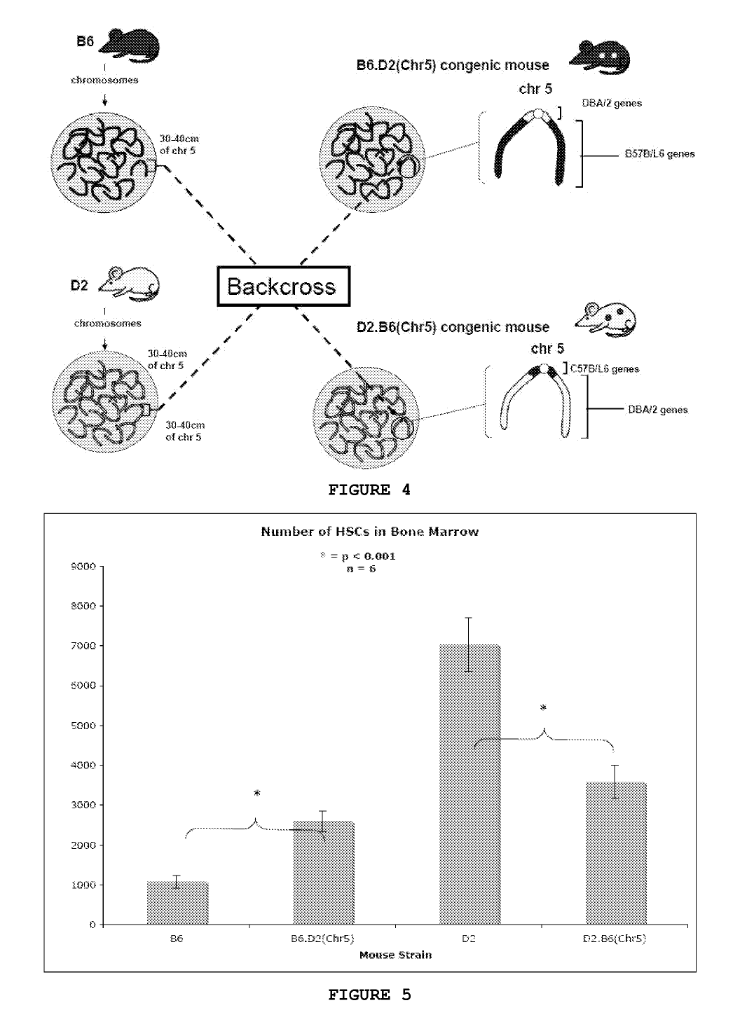

Generation of congenic mouse strains. Mouse strains congenic for the chromosome 5 QTL region were generated by marker-assisted crossing of the genomic interval carrying the QTL from B6 onto D2 (abbreviated D2.B6 Chr5) and vice versa (B6.D2 Chr5) using the ‘speed-congenic’ approach described previously (Markel et al. 1997, Wakeland et al. 1997). Both strains were bred to homozygosity at all loci, as verified by genotype analysis with 100 microsatellite markers distributed across the mouse genome. The consensus congenic interval ranges from 0-53 megabases on chromosome 5, encompassing the entire 95% confidence interval for the QTL (FIG. 4).

Validation of linkage analysis. In order to assess the physiological effect of the chromosome 5 QTL on stem cell number, CAFC assays were performed side-by-side on bone marrow cells from congenic and parental strains. Transfer of DBA alleles onto a B6 background resulted in a doubling of CAFCd28 cells, while B6 alleles on a DBA background reduced CAFCd28 number by half. The results of this analysis demonstrate that a gene or genes within the chromosome 5 QTL is/are sufficient to alter the size of the endogenous HSC pool (FIG. 5).

HSC Enrichment by Fluorescence Activated Cell Sorting (FACS). Mononucleated Bone Marrow Cells (MBMCs) were harvested from single cell suspensions of bone marrow using Ficoll gradient centrifugation. Viable, HSC enriched populations were sorted using Fluorescence Activated Cell Sorting (FACS) on a FacsVantage (Becton Dickinson, Franklin Lakes, N.J.) by negative selection for cells bearing lineage specific antigens. Lineage positive cells are labeled by a cocktail of biotinylated antibodies {CD5/Ly-1 (T-cells), CD45R/B220 (B-cells), CD11b/Mac-1(macrophages), CD8a/Ly2(T-cells), Gr-1/Ly-6G (granulocytes), and TER-119/Ly-76 (red cells)} and subsequently stained with Streptavidin-FITC. This enriched population is termed Lineage negative (Lin-). Where indicated, further enrichment was achieved by staining and selecting for cells that bear the Kit receptor (c-kit) and stem cell antigen-1 (Sca-1). These cells are referred to as LSK cells for Lineage negative, Sca-1 positive, and c-kit positive. Viability was determined by the absence of Propidium Iodide (PI) uptake.

RNA isolation. Total RNA was extracted from hematopoietic cells using a Quiagen RNeasy Mini Kit (Qiagen, Valencia, Calif.) according to manufacturer's instructions. This isolation method selects polyadenylated RNAs by an oligo (dT)-containing cellulose membrane. Isolated RNA is eluted in water and either 1) used directly for microarray analysis or 2) reversed transcribed for use in quantitative RT-PCR.

Affymetrix Microarray. Triplicate RNA samples were isolated from 500,000-800,000 Lin-cells from B6 and B6.DBA(Chr5) strains. Each of the six RNA samples were reverse transcribed, labeled with a biotin-conjugated probe and hybridized to a single Murine Genome 430 2.0 Affymetrix Microarray Chip (Affymetrix Inc., Santa Clara, Calif.) at the University of Kentucky Microarray Core Facility in Lexington, Ky. Expression was quantified with an Affymetrix GeneArray scanner, and average expression values for each strain were derived from the three replicate chips. Differences in the mean expression values between the congenic and background strain were compared for all 29,046 probesets that were detected in an least one strain. A T-test revealed that 1,340 were differentially expressed (determined by a p value of less than or equal to 0.05) between the strains, forty-one of which are located in the consensus congenic interval.

Illumina Micorarray. Triplicate RNA samples were isolated from 80,000-137,000 LSK cells of B6, DBA, B6.DBA(Chr5), and DBA.B6(Chr5) mice. Each of the twelve samples were reverse transcribed, purified, labeled, and hybridized to individual arrays on two Illumina Sentrix-Mouse 6 Whole Genome Expression Beadchips (Illumina Inc., San Diego, Calif.). Illumina expression arrays were processed on a fee-for-service basis at the Cincinnati Children's Hospital Medical Center Microarray Core Facility in Cincinnati, Ohio. Expression was quantified with an Ilumina Bead Scanner. Quality control and gene expression analysis were performed using Illumina BeadStudio v1.5.0.34. Average expression values were obtained for each of the four groups and normalized by the Rank Invariant normalization method. Differences in gene expression between congenic and parental strains were identified using the Illumina Custom differential expression algorithm and a p-value cutoff of 0.05 (Illumina diffscore of less than −13 or greater than 13). One-hundred and fifty-two differentially expressed genes were common to both congenic-parental strain comparisons and six are located in the 95% confidence interval for the chromosome 5 QTL.

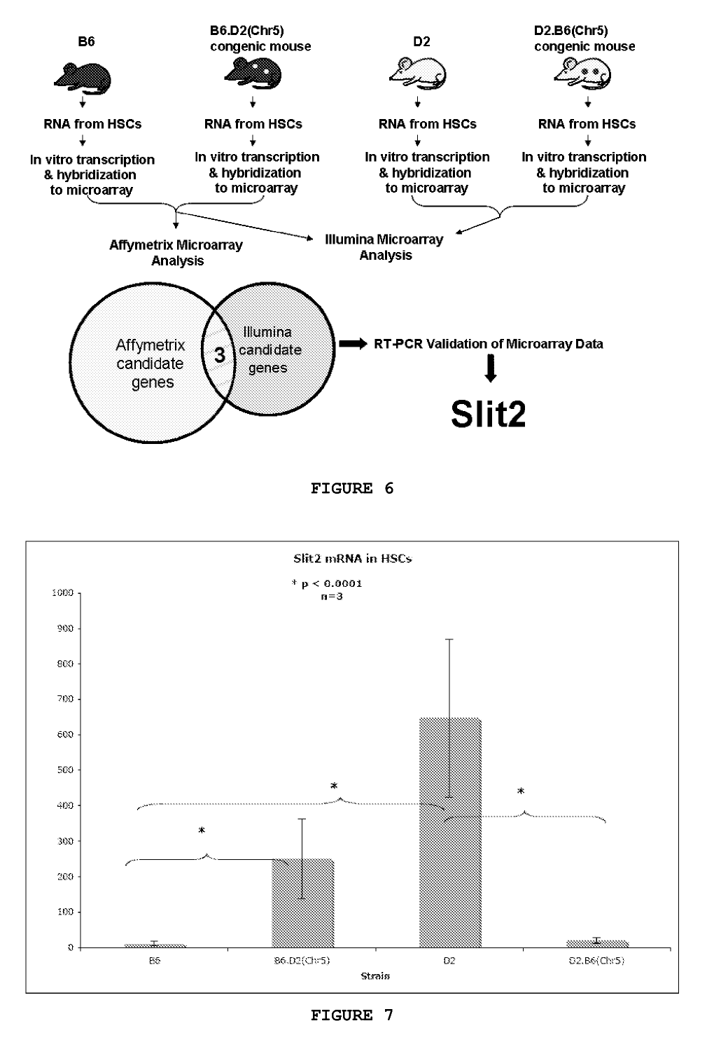

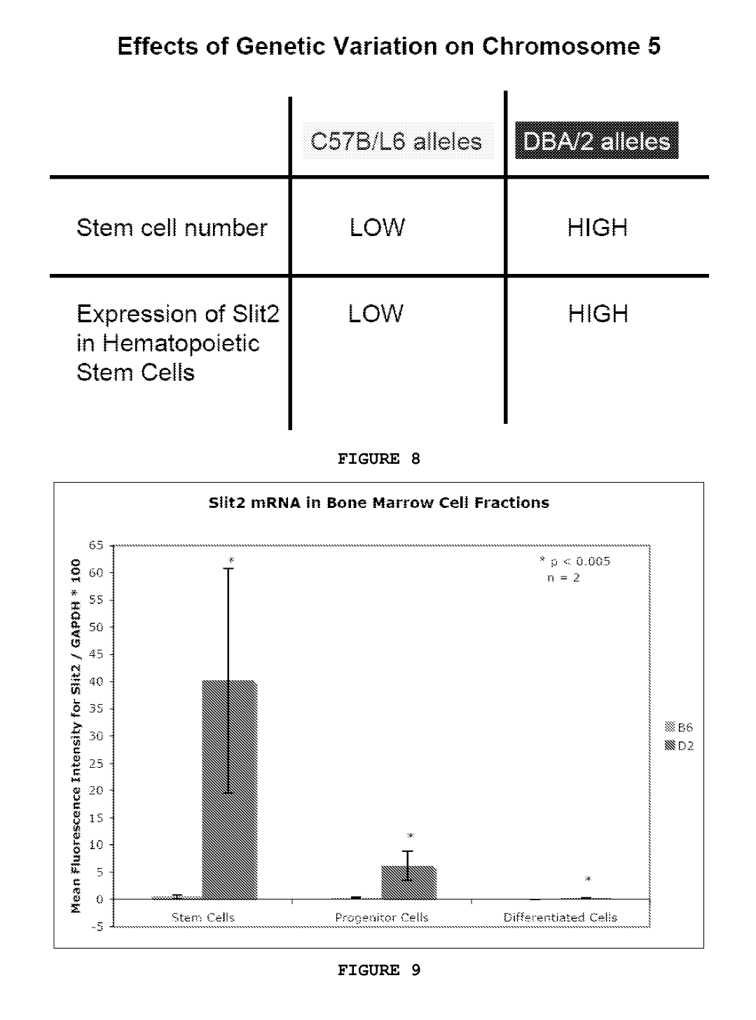

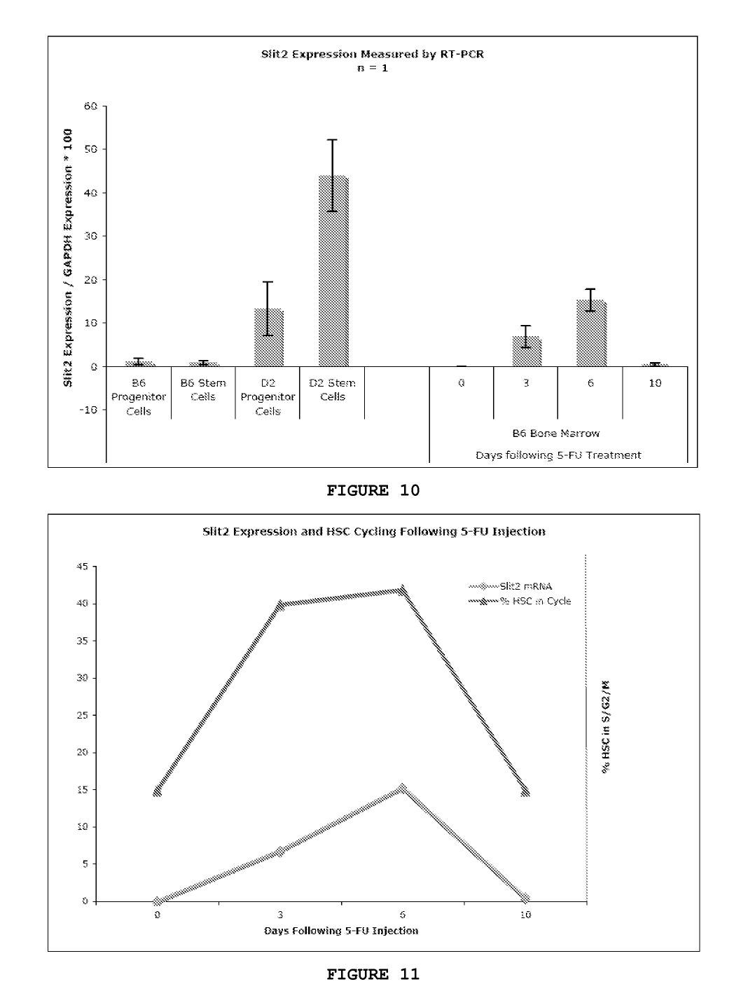

Selection of candidate genes from the microarray analysis. Three candidate genes were common to the list of 41 candidates identified in the Affymetrix microarray analysis and the six candidates identified by the Illumina microarray analysis (FIG. 6). These candidates, Slit2, QDPR (quinoid dihydropteridine reductase), and Gpr125 (G protein-coupled receptor 125) were further evaluated by qRT-PCR.

Quantitative Real Time-PCR (qRT-PCR). RNA isolated from LSK cells from each of the four strains was reverse transcribed using Taqman Reverse Transcription Reagents from Applied Biosystems (Foster City, Calif.) and stored at −20° C. PCR reactions were performed using Applied Biosystems primer and probe mixes for mouse Slit2 (PN 4331182), QDPR(PN 4331182) and GAPDH (PN 4308313) and TaqMan Universal PCR MasterMix (PN 4304437). Gene expression was analyzed by the ABI PRISM 7700 Sequence Detection System (Applied Biosystems). Expression of Slit2 and QDPR was determined by mean fluorescence intensity normalized to GAPDH. Only the Slit2 transcript was confirmed as being differentially expressed by RT-PCR. (For results of Slit2 RT-PCR see FIG. 7).

5-FU treatment. Six to sixteen-week old B6 mice were injected intra-peritoneally with 150 mg/kg total body weight of 5-fluorouracil (PN F6627-1G) (Sigma-Aldrich, Saint Louis, Mo.). Whole bone marrow cells were harvested from mice at 0, 3, 6 or 10 days following 5-FU, subjected to hypotonic lysis to eliminate red blood cells and used for RNA isolation and qRT-PCR analysis as described above (FIG. 9).

Data Analysis. Statistical comparisons between congenic and background strains were performed using a T-test assuming unequal variance unless tested for equal variance.

A number of embodiments of the disclosure have been described. Nevertheless, it will be understood that various modifications may be made without departing from the spirit and scope of the disclosure. Accordingly, other embodiments are within the scope of the following claims.

What is claimed is:

1. A composition comprising a basal medium and a Slit2 polypeptide or agonist thereof.

2. The composition of claim 1, further comprising serum.

3. The composition of claim 2, wherein the serum is allogeneic.

4. The composition of claim 2, wherein the serum is human.

5. The composition of claim 1, further comprising amino acids.

6. The composition of claim 5, wherein the amino acids are non-essential amino acids.

7. The composition of claim 1, further comprising a reducing agent.

8. The composition of claim 7, wherein the reducing agent is beta-mercaptoethanol.

9. The composition of claim 1, further comprising antibiotics and/or fungicides.

10. The composition of claim 1, further comprising a pyruvate salt.

11. The composition of claim 10, wherein the pyruvate salt is sodium pyruvate or potassium pyruvate.

12. The composition of claim 1, further comprising L-gluatmine.

13. A composition comprising basal medium supplemented with serum, non-essential amino acids, an anti-oxidant, a reducing agent, growth factors, a pyruvate salt and a Slit2 polypeptide, homolog or variant thereof.

14. The composition of claim 13, wherein the basal medium is DMEM.

15. A kit comprising a composition of claim 1 or 13.

16. A method of culturing stem cells, comprising:
contacting the stem cells with a composition of claim 1 or 13, wherein the stem cells grow and proliferate.

17. A method of treating a hematopoietic disease or disorder associated with reduced hematopoietic cells, comprising administering to a subject a Slit2 agonist, wherein the Slit2 agonist promotes hematopoietic stem cell proliferation and growth in the subject.

18. The method of claim 17, wherein the Slit2 agonist is a Slit2 polypeptide, a Slit2 agonistic antibody, a Slit2 peptidomimetic, a Slit2 agonistic polynucleotide and any combination thereof.

19. The method of claim 28, wherein the Slit2 agonistic polynucleotide comprises a heterologous polynucleotide that induces expression of Slit2 in the subject.

20. The method of claim 19, wherein the heterologous polynucleotide comprises a Slit2 polynucleotide.

21. A method of treating a hematopoietic disease or disorder associated with reduced hematopoietic cells comprising
isolating hematopoietic stem cells (HSCs);
culturing the HSCs in the presence of a Slit2 agonist under conditions wherein the HSCs proliferate a grow to obtain an expanded HSC population; and
administering the expanded HSC population to the subject.

22. The method of claim 21, wherein the HSCs are allogeneic to the subject.

23. The method of claim 21, wherein the HSCs are autologous to the subject.

24. A method of treating a cell proliferative disorder or disease in a subject, wherein the cell proliferative disorder or disease comprise hematopoietic cells, the method comprising:
contact the subject with a Slit2 antagonist, wherein the Slit2 antagonist reduces the biological activity or expression of Slit2.

25. The method of claim 24, wherein the Slit2 antagonist is selected from the group consisting of an antibody to a Slit2 receptor; an antibody to a Slit2 polypeptide; a soluble domain of a Slit2 receptor; a Slit2 antisense molecule; and any combination thereof.

26. A method for diagnosing a cell proliferative disease or disorder comprising:
measuring an amount of a Slit2 polypeptide or polynucleotide in a sample;
comparing the amount in the sample to a control amount, wherein an increase relative to the control is indicative of a cell proliferative disease or disorder.

27. The method of claim 26, wherein the measuring comprises using an antibody that specifically binds to a Slit2 polypeptide.

28. The method of claim 26, wherein the measuring comprises an oligonucleotide probe or primer pair that hybridize to a Slit2 polynucleotide.

29. A pharmaceutical composition comprising a Slit2 agonist and a pharmaceutically acceptable carrier.

30. A pharmaceutical composition comprising a Slit2 antagonist and a pharmaceutically acceptable carrier.

31. The pharmaceutical composition of claim 30, wherein the Slit2 antagonist is detectably labeled.


SEQUENCE LISTING (14 sequences)

SEQ ID NO: 1 - DNA, 4950 bp, Homo sapiens
  CDS (205)..(4794)
cagagcagggtggagagggcggtgggaggcgtgtgcctgagtgggctctactgccttgttccatattattttgtgcacattttccctggcactctgggttgctagccccgccgggcactgggcctcagacactgcgcggttccctcggagcagcaagctaaagaaagcccccagtgccggcgaggaaggaggcggcggggaaagatgcgcggcgttggctggcagatgctgtccctgtcgctggggttagtgctggcgatcctgaacaaggtggcaccgcaggcgtgcccggcgcagtgctcttgctcgggcagcacagtggactgtcacgggctggcgctgcgcagcgtgcccaggaatatcccccgcaacaccgagagactggatttaaatggaaataacatcacaagaattacgaagacagattttgctggtcttagacatctaagagttcttcagcttatggagaataagattagcaccattgaaagaggagcattccaggatcttaaagaactagagagactgcgtttaaacagaaatcaccttcagctgtttcctgagttgctgtttcttgggactgcgaagctatacaggcttgatctcagtgaaaaccaaattcaggcaatcccaaggaaagctttccgtggggcagttgacataaaaaatttgcaactggattacaaccagatcagctgtattgaagatggggcattcagggctctccgggacctggaagtgctcactctcaacaataacaacattactagactttctgtggcaagtttcaaccatatgcctaaacttaggacttttcgactgcattcaaacaacctgtattgtgactgccacctggcctggctctccgactggcttcgccaaaggcctcgggttggtctgtacactcagtgtatgggcccctcccacctgagaggccataatgtagccgaggttcaaaaacgagaatttgtctgcagtggtcaccagtcatttatggctccttcttgtagtgttttgcactgccctgccgcctgtacctgtagcaacaatatcgtagactgtcgtgggaaaggtctcactgagatccccacaaatcttccagagaccatcacagaaatacgtttggaacagaacacaatcaaagtcatccctcctggagctttctcaccatataaaaagcttagacgaattgacctgagcaataatcagatctctgaacttgcaccagatgctttccaaggactacgctctctgaattcacttgtcctctatggaaataaaatcacagaactccccaaaagtttatttgaaggactgttttccttacagctcctattattgaatgccaacaagataaactgccttcgggtagatgcttttcaggatctccacaacttgaaccttctctccctatatgacaacaagcttcagaccatcgccaaggggaccttttcacctcttcgggccattcaaactatgcatttggcccagaacccctttatttgtgactgccatctcaagtggctagcggattatctccataccaacccgattgagaccagtggtgcccgttgcaccagcccccgccgcctggcaaacaaaagaattggacagatcaaaagcaagaaattccgttgttcagctaaagaacagtatttcattccaggtacagaagattatcgatcaaaattaagtggagactgctttgcggatctggcttgccctgaaaagtgtcgctgtgaaggaaccacagtagattgctctaatcaaaagctcaacaaaatcccggagcacattccccagtacactgcagagttgcgtctcaataataatgaatttaccgtgttggaagccacaggaatctttaagaaacttcctcaattacgtaaaataaactttagcaacaataagatcacagatattgaggagggagcatttgaaggagcatctggtgtaaatgaaatacttcttacgagtaatcgtttggaaaatgtgcagcataagatgttcaagggattggaaagcctcaaaactttgatgttgagaagcaatcgaataacctgtgtggggaatgacagtttcataggactcagttctgtgcgtttgctttctttgtatgataatcaaattactacagttgcaccaggggcatttgatactctccattctttatctactctaaacctcttggccaatccttttaactgtaactgctacctggcttggttgggagagtggctgagaaagaagagaattgtcacgggaaatcctagatgtcaaaaaccatacttcctgaaagaaatacccatccaggatgtggccattcaggacttcacttgtgatgacggaaatgatgacaatagttgctccccactttctcgctgtcctactgaatgtacttgcttggatacagtcgtccgatgtagcaacaagggtttgaaggtcttgccgaaaggtattccaagagatgtcacagagttgtatctggatggaaaccaatttacactggttcccaaggaactctccaactacaaacatttaacacttatagacttaagtaacaacagaataagcacgctttctaatcagagcttcagcaacatgacccagctcctcaccttaattcttagttacaaccgtctgagatgtattcctcctcgcacctttgatggattaaagtctcttcgattactttctctacatggaaatgacatttctgttgtgcctgaaggtgctttcaatgatctttctgcattatcacatctagcaattggagccaaccctctttactgtgattgtaacatgcagtggttatccgactgggtgaagtcggaatataaggagcctggaattgctcgttgtgctggtcctggagaaatggcagataaacttttactcacaactccctccaaaaaatttacctgtcaaggtcctgtggatgtcaatattctagctaagtgtaacccctgcctatcaaatccgtgtaaaaatgatggcacatgtaatagtgatccagttgacttttaccgatgcacctgtccatatggtttcaaggggcaggactgtgatgtcccaattcatgcctgcatcagtaacccatgtaaacatggaggaacttgccacttaaaggaaggagaagaagatggattctggtgtatttgtgctgatggatttgaaggagaaaattgtgaagtcaacgttgatgattgtgaagataatgactgtgaaaataattctacatgtgtcgatggcattaataactacacatgcctttgcccacctgagtatacaggtgagttgtgtgaggagaagctggacttctgtgcccaggacctgaacccctgccagcacgattcaaagtgcatcctaactccaaagggattcaaatgtgactgcacaccagggtacgtaggtgaacactgcgacatcgattttgacgactgccaagacaacaagtgtaaaaacggagcccactgcacagatgcagtgaacggctatacgtgcatatgccccgaaggttacagtggcttgttctgtgagttttctccacccatggtcctccctcgtaccagcccctgtgataattttgattgtcagaatggagctcagtgtatcgtcagaataaatgagccaatatgtcagtgtttgcctggctatcagggagaaaagtgtgaaaaattggttagtgtgaattttataaacaaagagtcttatcttcagattccttcagccaaggttcggcctcagacgaacataacacttcagattgccacagatgaagacagcggaatcctcctgtataagggtgacaaagaccatatcgcggtagaactctatcgggggcgtgttcgtgccagctatgacaccggctctcatccagcttctgccatttacagtgtggagacaatcaatgatggaaacttccacattgtggaactacttgccttggatcagagtctctctttgtccgtggatggtgggaaccccaaaatcatcactaacttgtcaaagcagtccactctgaattttgactctccactctatgtaggaggcatgccagggaagagtaacgtggcatctctgcgccaggcccctgggcagaacggaaccagcttccacggctgcatccggaacctttacatcaacagtgagctgcaggacttccagaaggtgccgatgcaaacaggcattttgcctggctgtgagccatgccacaagaaggtgtgtgcccatggcacatgccagcccagcagccaggcaggcttcacctgcgagtgccaggaaggatggatggggcccctctgtgaccaacggaccaatgacccttgccttggaaataaatgcgtacatggcacctgcttgcccatcaatgcgttctcctacagctgtaagtgcttggagggccatggaggtgtcctctgtgatgaagaggaggatctgtttaacccatgccaggcgatcaagtgcaagcacgggaagtgcaggctttcaggtctggggcagccctactgtgaatgcagcagtggatacacgggggacagctgtgatcgagaaatctcttgtcgaggggaaaggataagagattattaccaaaagcagcagggctatgctgcttgccaaacaaccaagaaggtgtcccgattagagtgcagaggtgggtgtgcaggagggcagtgctgtggaccgctgaggagcaagcggcggaaatactctttcgaatgcactgacggctcctcctttgtggacgaggttgagaaagtggtgaagtgcggctgtacgaggtgtgtgtcctaaacacactcccggcagctctgtctttggaaaaggttgtatacttcttgaccatgtgggactaatgaatgcttcatagtggaaatatttgaaatatattgtaaaatacagaacagacttatttttattatgagaataaagactttttttctgcatttg

SEQ ID NO: 2 - protein, 1529 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGCTRCVS

SEQ ID NO: 3 - protein, 5 aa, Artificial Sequence
  Artificial peptide sequence
GGGGS

SEQ ID NO: 4 - protein, 6 aa, Artificial Sequence
  Artificial peptide sequence
GGGGSX

SEQ ID NO: 5 - protein, 12 aa, Artificial Sequence
  Artificial Peptide Sequence
GKSSGSGSESKS

SEQ ID NO: 6 - protein, 14 aa, Artificial Sequence
  Artificial Peptide Sequence
GSTSGSGKSSEGKG

SEQ ID NO: 7 - protein, 18 aa, Artificial Sequence
  Artificial Peptide Sequence
GSTSGSGKSSEGSGSTKG

SEQ ID NO: 8 - protein, 18 aa, Artificial Sequence
  Artificial Peptide Sequence
GSTSGSGKPGSGEGSTKG

SEQ ID NO: 9 - protein, 14 aa, Artificial Sequence
  Artificial Peptide Sequence
EGKSSGSGSESKEF

SEQ ID NO: 10 - protein, 5 aa, Artificial Sequence
  Artificial Peptide Sequence
KKKRK

SEQ ID NO: 11 - protein, 26 aa, Artificial Sequence
  Artificial Peptide Sequence
MLRTSSLFTRRVQPSLFRNILRLQST

SEQ ID NO: 12 - protein, 4 aa, Artificial Sequence
  Artificial Peptide Sequence
KDEL

SEQ ID NO: 13 - protein, 4 aa, Artificial Sequence
  Artificial Peptide Sequence
CAAX

SEQ ID NO: 14 - protein, 4 aa, Artificial Sequence
  Artificial Peptide Sequence
CCXX